(19) United States
(12) Patent Application Publication
(10) Pub. No.: US 2007/0122377 A1
(43) Pub. Date: May 31, 2007

(54) Compositions and methods of treatment

(76) Inventors: Simon Geoffrey Best, Edinburgh (GB); Rodney William Kelly, Edinburgh (GB)

Correspondence Address: NIXON PEABODY LLP - PATENT GROUP, CLINTON SQUARE, P.O. BOX 31051, ROCHESTER, NY, 14603-1051, US

(21) Appl. No.: 10/576,437

(22) PCT Filed: Oct. 19, 2004
(86) PCT No.: PCT/GB04/04412

§ 371 Date: Nov. 3, 2006

(30) Foreign Application Priority Data
Oct. 21, 2003 (GB) 0324523.0

(51) Int. Cl. A61K 38/19; A61K 31/7076

(52) U.S. Cl. 424/85.1; 514/48

(57) ABSTRACT

A method of inducing tolerance to a therapeutic cell in a patient who is to be administered subsequently a therapeutic amount of the said therapeutic cell or a precursor thereof, the method comprising administering to the patient (a) a tolerising cell sharing the same antigenic characteristics as the therapeutic cell, or an antigen found thereon or a derivative of said antigen, and (b) an agent which raises the effective cAMP concentration in a monocyte cell. A method of reducing the risk of rejection of a transplant in a patient in need of transplantation of a therapeutic cell for cell or tissue regeneration, the method comprising administering to the patient prior to the transplant (a) a tolerising cell sharing the same antigenic characteristics as the therapeutic cell which therapeutic cell is, or is able to differentiate into, the cell or tissue to be regenerated, or an antigen found thereon or a derivative of said antigen, and (b) an agent which raises the effective cAMP concentration in a monocyte cell. A method of treating a patient in need of cell or tissue regeneration the method comprising administering to the patient (a) a tolerising cell sharing the same antigenic characteristics as the therapeutic cell to be administered subsequently which therapeutic cell is, or is able to differentiate into, the cell or tissue to be regenerated, or an antigen found thereon or a derivative of said antigen, (b) an agent which raises the effective cAMP concentration in a monocyte cell in an amount to induce tolerance to the said therapeutic cell, and subsequently administering to the patient (c) a therapeutic amount of the said therapeutic cell. Preferably, the agent which raises the effective cAMP concentration in a monocyte cell is a prostaglandin. Preferably it is used in combination with GMCSF or a derivative thereof.

The present invention relates to therapeutic compositions, methods and uses; in particular it relates to methods for treating degenerative diseases in a patient.

Many serious medical conditions, such as Type I diabetes, osteoarthritis, rheumatoid arthritis, multiple sclerosis, heart failure, stroke, burns, osteoporosis, bone fractures, Parkinson's disease and spinal chord injury, are due to the failure of or damage to tissue or a cell type within a patient due to disease or trauma. These can be considered degenerative diseases, some of which are associated with aging and where cells are unable to repair themselves or be replaced. Current treatments are limited to being palliative, delaying progression, and tissue function is typically not restored. Recent breakthroughs in the isolation, expansion and controlled differentiation of human adult and embryonic stem cells and the restoration of normal tissue function in animal models of degenerative disease following experimental transplantation have opened up the possibility of a new major field of regenerative medicine. New procedures are being developed to correct the failure of or damage to the tissue or cell type concerned by introducing into the patient cells which are able to take the place and function of the failed or damaged tissue or cells. In some cases, these may be exactly the same type of cells that are failed or damaged. In other cases, the introduced cells will be precursor cells of the tissue or cell type to be replaced, which are able to differentiate into the desired tissue or cell type at the site of disease or injury. In some cases, cells will be introduced at the precise site of disease or trauma; in others, cells will be introduced into portal veins, ventricles or elsewhere in the vasculature, circulatory or lymphatic systems to facilitate migration to the site of disease or trauma.

An example of this approach is in relation to Parldnson's disease, which is a very common neurodegenerative disorder that affects more than 2% of the population over 65 years of age. Parkinson's disease is caused by a progressive degeneration and loss of dopamine-producing neurons, which leads to tremor, rigidity and hypokinesia (abnormally decreased mobility). A recent study has shown that mouse embryonic stem cells can differentiate into dopamine-producing neurons by introducing the Nurrl gene. When transplanted into the brains of a rat model of Parkinson's disease, these stem cell-derived dopamine-producing neurons reinnervated the brains of the rat Parkinson models, released dopamine and improved motor function.

A further example of the approach is the use of cardiomyocytes or bone marrow stem cells to repair damage to heart muscle tissue for example in chronic heart disease or after an infarction. A still further example is the use of oligodendrocytes for repairing damage to the spinal chord. A yet still further example is the use of derivatives of human embryonic stem cells which are able to differentiate into insulin-producing cells that can be used in transplantation therapy to treat Type I diabetes.

Useful information on stem cells and their use in regenerative medicine may be found on the National Institutes of Health web site, for example at http://stemcells.nih.gov. In addition, the potential of stem cells is reviewed by Pfendler & Kawase (2003) Obstetrical & Gynecological Survey 58, 197-208, incorporated herein by reference.

One of the most important applications of human stem cells, therefore, is the generation of cells and tissues that can be used for cell-based therapies. Today, donated organs and tissues are often used to replace failing or destroyed tissue, but the need for transplantable tissues and organs far outweighs the available supply, hence the great interest in cell-based, particularly stem cell-based therapy.

To realise the potential of cell-based therapies for such pervasive and debilitating diseases as those discussed above, it is necessary for the cells to survive in the patient after transplantation. Unless the cell used in the therapy, such as the stem cell, is derived from the patient, it is highly likely that the patient will raise an immune response to it, thereby reducing the chances of it being rejected by the patient's immune system, and increasing the likelihood that immunosuppressive drugs will have to be used. This is because the introduced cell is considered to be “foreign” by the patient's immune system because of the presence of “foreign” antigens on the cell. Indeed, this is the conclusion reached in relation to human embryonic stem cells, where Drukker et al (2002) Proc. Natl. Acad Sci. USA 99, 9864-9869 notes S that these cells can express high levels of MHC-I proteins and thus may be rejected on transplantation. However, in Bell (2002) Nature Reviews Immunology 2, 75 there is a suggestion that embryonic stem cells may survive in allogeneic hosts in the absence of host conditioning.

Ways to reduce the possibility of undesirable immune responses and rejection of cells used in therapy have been suggested. For example, a “master” embryonic stem cell line may be produced in which the major histocompatibillty complex (MHC) genes have been genetically modifying or knocked out. However, this may be technically difficult to achieve and, if accomplished, could expose the recipient of the MHC null transplant to new risks of infectious disease and/or cancer. An alternative strategy that has been suggested is to introduce the recipient's MHC genes into the embryonic stem cell through targeted gene transfer, but because of the differences among MHC proteins among individuals, the donor stem cells may be recognised as non-self by the patient's immune system and trigger graft versus host disease (ie destruction by cytotoxic T cells) and ultimately rejection. Furthermore, this approach is also technically demanding and complex.

An organism's immunity to an antigen arises as a consequence of a first encounter with the antigen and the subsequent production of immunoglobulin molecules, for example, antibodies, capable of selectively binding that antigen. In addition, the immune response is controlled by T cells which may be antigen specific. A large proportion of the memory T-cell population (8-10%) will recognise MHC antigens. Immunity allows the rapid recruitment, usually by stimulating an inflammatory response, of cells which can dispose of the foreign antigen. Under certain circumstances, the immune system does not produce an immune response against antigens due to a mechanism called “tolerance”. For example, an immune system can normally discriminate against foreign antigens and constituents of the organism itself, due to a mechanism whereby all T and B lymphocytes which could potentially produce antibodies to constituents of the organism itself (“self antigens”) are destroyed during development, thereby removing the organism's capacity to produce antibodies directed to a self antigen.

One way that has been suggested of tolerising a patient who is undergoing a cell transplant is to have pre-tolerised the patient to the MHC antigens of the “master” embryonic stem cell line from which the cell or tissue for transplantation will be derived. This requires a procedure somewhat akin to a bone marrow transplant, and so certainly is invasive and requires some degree of immunosuppression.

The inventors now describe a much simpler method for inducing tolerance in (or “pre-tolerising”) a patient to a cell or tissue which “regenerates” failed or damaged cells or tissues in the patient by producing a tolerant environment in the patient into which a cell is introduced which is a precursor of the cell or tissue to be generated. In particular, the tolerant environment into which the precursor cell is introduced is created using an agent which is able to raise the effective cAMP concentration in a monocyte cell, such as a prostaglandin, preferably in combination with granulocyte-macrophage colony stimulating factor (GMCSF) or a derivative thereof. Typically, the prostaglandin may also be used in combination with a phosphodiesterase inhibitor.

It has been found that there is a synergistic effect between prostaglandin and a phosphodiesterase (PDE) inhibitor on the release of interleukin-10 (IL-10) from cells of the immune system. Furthermore, it has been found that there is a marked stimulation of IL-10 and inhibition of interleukin-12 (IL-12) in cells of the immune system when a prostaglandin and a PDE inhibitor are used in combination. In the presence of a PDE inhibitor, the stimulation of IL-10 by both PGE and 19-hydroxy PGE was increased strikingly, resulting in a tolerising environment.

PDE inhibitors such as Rolipram are known to raise cAMP and IL-10 levels in monocyte/macrophages stimulated with the bacterial coat product lipopolysaccharide (LPS) (Strassman et al (1994) J. Exp. Med. 180: 2365-70; Kraan et al (1995) J. Exp. Med. 181: 775-9; Kambayashi et al (I995) J. Immunol. 155: 4909-16).

It has also been shown that an increase in PDE activity follows both PGE and 19-hydroxy PGE application. This is a direct negative feedback to reduce the effect of the stimulus. Use of a PGE and a PDE inhibitor increases PDE message even further, but then the synthesised phosphodiesterase is nullified by the presence of the inhibitor.

The principal receptors for prostaglandin E2 (PGE2) are the EP2 and EP4 sub-types; however, other receptor sub-types exist (namely EP1 and EP3). EP2 and EP4 receptors couple with adenylcyclase and use elevated cAMP as the messenger system. The levels of cAMP in tissue are governed both by synthesis and by catabolism by PDE. PDE can be blocked by specific inhibitors. The inventors is believe, but without being bound by any theory, that the administration of a PDE inhibitor will enhance the effect of a prostaglandin or agonist thereof in inducing tolerance to a precursor cell (or an antigen found thereon or a derivative thereof) that is administered to a patient. Thus, the inventors believe, but without being bound by any theory, that the effect of a prostaglandin or agonist thereof (such as PGE) acting on its EP2 and EP4 receptors is to stimulate cAMP and the addition of the PDE inhibitor provides a synergistic action on monocytes and macrophages resulting in a reduction in the immune and/or inflammatory response which is greater than the effect of the sum of the same amount of either prostaglandin or agonist thereof or PDE inhibitor administered alone.

It has also been found that there is a marked stimulation of IL-10 in cells of the immune system when an agent which raises the effective cAMP concentration in monocyte cells, such as a prostaglandin, and granulocyte-macrophage colony stimulating factor (GMCSF) are used in combination. It has been found that there is a synergistic effect between a prostaglandin and GMCSF on the release of IL-10 from cells of the immune system; in the presence of GMCSF the stimulation of IL-10 by both prostaglandin E (PGE) and 19-hydroxy PGE was increased strikingly, resulting in a tolerising environment. In other words, it is believed that GMCSF and an agent that raises the effective cAMP concentration in a monocyte cell, such as a prostaglandin, polarises monocytes into a phenotype characterised by increased IL-10 release. Similarly, in the presence of GMCSF the stimulation of IL-10 expression by forskolin is increased strikingly, and in a synergistic way compared to forskolin or GMCSF alone. Not only is the cell directed to a pro-tolerance phenotype but this is also accompanied by enhanced production of granulysin, an anti-microbial agent. In addition, the effects of PGE and GMCSF are prolonged and continue after the removal of these agents thus the cell is selectively differentiated.

GMCSF has an important role in granulocyte and macrophage lineage maturation GMCSF has been proposed as both a treatment agent and a target for treatment. Recombinant human GMCSF has been used to treat some cancers and to promote haematopoietic reconstitution following bone marrow transplantation (Leukine® Package Insert Approved Text, February 1998, and Buchsel et al (2002) Clin. J. Oncol. Nurs. 6(4): 198-205). By contrast, other recent reports describe GMCSF as being a potential target for treatment of inflammatory and immune diseases (Hamilton (2002) Trends Immunol 23(8): 403-8) and asthma Ritz et al (2002) Trends Immunol 23(8): 396-402).

In diseases resulting from an aberrant or undesired immune response there is often a deficiency in IL-10. This deficiency in IL-10 may be detrimental to the development of usefull T helper cells, particularly type-2 T helper cells; a preponderance of type 1 T helper cells over type 2 T helper cells is thought to be characteristic of autoimmune disease. Thus, stimulation of IL-10 production is believed to induce a tolerising environment for T cell priming. In addition, a high IL-10 environment will act on an antigen presenting cell (typically a dendritic cell) to ensure regulatory T cell formation, creating a regulatory T cell that is specific for the antigen presented.

Without being bound by theory, the inventor believes that a combination of GMCSF and an agent which raises the effective cAMP concentration in a monocyte cell, such as a prostaglandin or forskolin, will also decrease IL-12 levels, which would be expected to enhance the effects of the invention. It has been shown that the combination of a prostaglandin and GMCSF increases the expression of both IL-10 and COX-2, and that the combination of a forskolin and GMCSF synergistically increases the level of IL-10 in a monocyte cell. The decrease in IL-12 levels may therefore arise through the direct inhibition of IL-12 by IL-10 (Harizi et al (2002) J. Immunol. 168, 2255-2263) or through an IL-10 independent pathway that depends on COX-2 induction (Schwacha et al (2002) Am. J. Physiol. Cell Physiol. 282, C263-270).

It has also been shown that PGE and GMCSF reduce levels of participants in antigen presentation such as class II transactivator (CIITA) and MHC class II (as shown in Example 1). This change in phenotype is accompanied by enhanced expression of granulysin which has antimicrobial, including antiviral, properties (Krensky (2000) Biochem. Pharmacol. 59, 317-320) and is normally thought of as a product of activated T cells that mediates antiviral activity that lyses infected cells (Hata et al (2001) Viral Immunol. 14, 125-133; Ochoa et al (2001) Nature Medicine 7, 174-179; Smyth et al (2001) J. Leukoc. Biol. 70, 18-29). The increased expression of granulysin is believed to be an important consequence of the present invention, as the increase in innate defence molecules may compensate for the compromise of the adaptive immune system that accompanies tolerance induction.

In addition, it has been shown that a combination of PGE and GMCSF increases the expression of COX-2, CD86, CD14. COX-2 is believed to be involved in maintaining the tolerant phenotype after removal of the prostaglandin and GMCSF (as is shown in Examples 2 and 3), and both CD14 and CD86 are differentiation markers and are evidence of a more differentiated state.

The inventors now propose inducing tolerance to a cell in a patient by the use of an agent which raises the effective cAMP concentration in a monocyte cell in order to induce a tolerising environment in the patient, and by administering the cell or a precursor thereof or an antigen found thereon or a derivative of said antigen to the patient, such that tolerance to the cell is induced in the patient. By this process the patient is also made tolerant to a therapeutic cell which has the same antigenic characteristics as the cell used for tolerisation.

As far as the inventors are aware, no-one has suggested the use of this system of generating tolerance in connection with cell based therapies or its use in cellular transplants for treating degenerative disease.

The listing or discussion of a prior-published document in this specification should not necessarily be taken as an acknowledgement that the document is part of the state of the art or is common general knowledge.

A first aspect of the invention provides a method of inducing tolerance to a therapeutic cell in a patient who is to be administered subsequently a therapeutic amount of the said therapeutic cell or a precursor thereof, the method comprising administering to the patient (a) a tolerising cell sharing the same antigenic characteristics as the therapeutic cell, or an antigen found thereon or a derivative of said antigen, and (b) an agent which raises the effective cAMP concentration in a monocyte cell.

By inducing tolerance, we include the meaning that when the patient is subsequently administered the therapeutic cell or a precursor thereof, a greatly reduced or non-damaging immune response with respect to the therapeutic cell is experienced by the patient compared to a patient who has not been pretolerised. The well known mixed lymphocyte test may be used to determine whether a patient has been pretolerised. Alternatively, loss of the 6C10 marker (as described in Maier et al (1998) Proc. Natl. Acad. Sci. USA 95, 4499) may be used.

It will be appreciated that inducing tolerance in, or pretolerising, the patient is beneficial in those patients who will subsequently be administered a therapeutic amount of the therapeutic cells when undergoing transplantation for the purpose of repairing or regenerating damaged cells or tissue. A therapeutic amount of the cells is the amount which is needed to be administered to the patient in order to achieve a beneficial effect in terms of satisfying the need of the patient, for example in combating a degenerative disease or disorder. The therapeutic cells typically are used to repair or regenerate failed or damaged cells or tissues.

It will be appreciated that a benefit of inducing tolerance (or pretolerising) is that the chances of an adverse reaction on subsequent transplantation of the therapeutic cells is reduced.

By “sharing the same antigenic characteristics” we include the meaning that the tolerising cell has sufficient cell surface antigens, typically MHC antigens, in common with the therapeutic cell that administration of the tolerising cell to the patient in a tolerising environment leads to tolerance to the subsequent administration of the therapeutic cell so that the likelihood of rejection of the therapeutic cell is reduced compared to when no tolerisation is used.

Typically, the tolerising cell and therapeutic cell are syngeneic and express substantially the same antigens.

A second aspect of the invention provides a method of reducing the risk of rejection of a transplant in a patient in need of transplantation of a therapeutic cell for cell or tissue regeneration, the method comprising administering to the patient prior to the transplant (a) a tolerising cell sharing the same antigenic characteristics as the therapeutic cell which therapeutic cell is, or is able to differentiate into, the cell or tissue to be regenerated, or an antigen found thereon or a derivative of said antigen, and (b) an agent which raises the effective cAMP concentration in a monocyte cell.

A third aspect of the invention provides a method of treating a patient in need of cell or tissue regeneration the method comprising administering to the patient (a) a tolerising cell sharing the same antigenic characteristics as the therapeutic cell to be administered subsequently which therapeutic cell is, or is able to differentiate into, the cell or tissue to be regenerated, or an antigen found thereon or a derivative of said antigen, (b) an agent which raises the effective cAMP concentration in a monocyte cell in an amount to induce tolerance to the said therapeutic cell, and subsequently administering to the patient (c) a therapeutic amount of the said therapeutic cell.

Although the invention may be used in connection with any mammal, including domestic and farm mammals such as cat, dog, horse, sheep, cow and the like, typically the patient is a human.

The diseases where the methods of the invention may be used include degenerative diseases or disorders by which we include diabetes, where insulin-producing cells fail; stroke, Parkinson's disease, ALS (Lou Gehrig's disease) and spinal cord injury, where nerve cells fail; heart attack, cardiac ischaemia and congestive heart failure, where heart muscle cells fail; cirrhosis and hepatitis, where liver cells fail; certain cancers and immunodeficiency, where blood, bone marrow or haematopoietic cells fail; osteoporosis, where bone cells fail; osteoarthritis, where cartilage cells fail; burns and wounds, where skin cells fail; muscular dystrophy, where skeletal muscle cells fail; age-related macular degeneration where retinal cells fail; and multiple sclerosis, where myelin is destroyed (Schwann cells fail).

It will be appreciated that in preferred embodiments of the practice of the methods of the invention there are two distinct points at which the patient is administered a cell. The first is in the context of tolerising the patient, and the second is in the context of administering a cell in therapeutic amounts once the patient has been tolerised to the cell.

The first cell (“tolerising cell”) may be any suitable cell which shares the same antigenic characteristics as the second cell (“therapeutic cell”). Thus, typically and preferably, the tolerising cell and the therapeutic cell are derived from the same embryonic stem cell (and therefore have the same antigenic characteristics). It will be appreciated that the cells in question are foreign to the patient to be treated since if they are from the patient to be treated, there is no need for pretolerisation.

Typically, the tolerising cell is a cell which has good expression of MHC molecules, since these molecules are the principle antigenic determinants relevant to transplant rejection. Good expression of other antigens that may be relevant to transplant rejection is also desirable. The tolerising cell may be the same as the therapeutic cell; however, it may also be a precursor of the therapeutic cell ie a cell which is capable of differentiating into the therapeutic cell and which is already committed to differentiate into the therapeutic cell. Thus, the tolerising cell is not a pluripotent cell (ie one which can differentiate into any cell) since such cells may spontaneously form teratomas and are not suitable for administration to a patient. If the tolerising cell is not the same as the therapeutic cell it is preferred if it is a precursor cell which is one or two or three or more stages less differentiated (on the same differentiation pathway) as the therapeutic cell.

In connection with the use of adult stem cells as the therapeutic cells, it may be advantageous to use cells derived from the peripheral blood of the donor as the tolerising cells. Suitable cells include peripheral blood leukocytes which have good expression of MHC antigens. Alternatively, stem cells isolated from peripheral blood (eg a monocyte-derived subset; Zhao et al (2003) Proc. Natl. Acad. Sci. USA 100, 2426-2431) may be differentiated using, for example, EGF to give an epithelial phenotype, and used as tolerising cells.

The therapeutic cell is any suitable therapeutic cell. It may be a cell which is the same as the cell type which is damaged or diseased in the patient or one which is able to generate tissue which is damaged or diseased in the patient. Preferably, the cell is a precursor of the cell or tissue to be replaced or repaired, which is able to differentiate into the cell or tissue which is to be replaced or repaired. The cell is one which is already committed to differentiate into the cell or tissue to be replaced or repaired, and is not pluripotent (since such cells may cause the production of teratomas as discussed above).

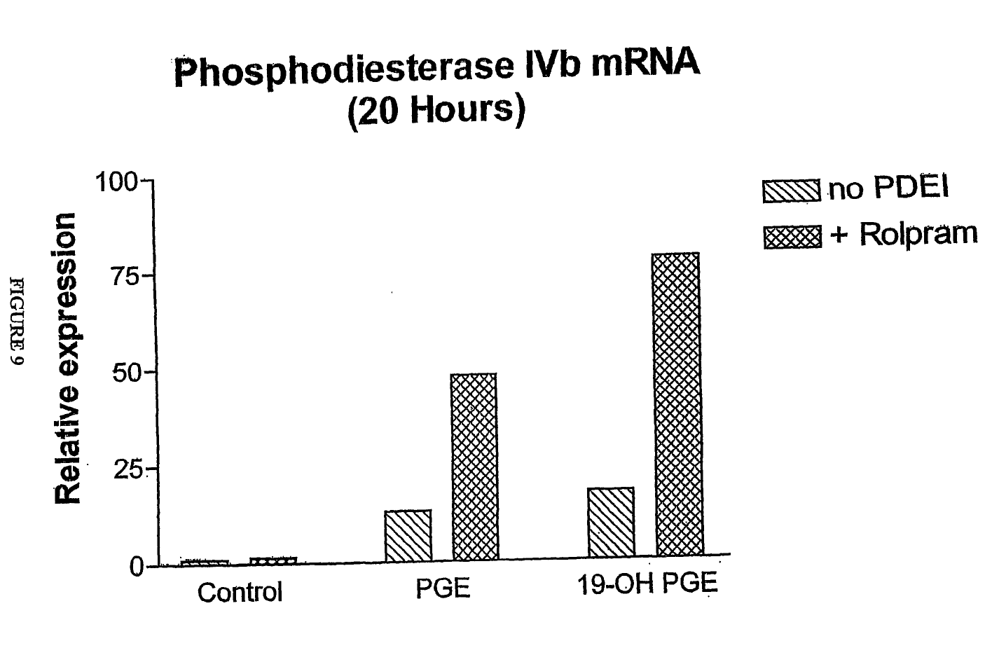

The therapeutic cell (and therefore consequently the tolerising cell) is chosen by reference to the disease or disorder to be treated. Thus, typically, the therapeutic cell is, or is able to differentiate into, the cell or tissue which is to be regenerated in treating the disease or disorder. For example, in relation to precursor cells, in the case of diabetes, the precursor cell is one which is able to differentiate into an insulin-producing cell; in the case of congestive heart failure, the precursor cell is one which is able to differentiate into heart muscle cells; in the case of Parkinson's disease, the precursor cell is one which is able to differentiate into a suitable nerve cell; and so on. Suitable precursor cells which are able to differentiate into a type of cell or tissue which is used to replace the function of a failed or damaged cell or tissue in a degenerative disease or disorder are known in the art. FIG. 10 describes stem cell lineages for human pluripotent stem cells (hPSCs), and suitable precursor cells, including stem cells (but not pluripotent stem cells as discussed above), may be selected by reference to this figure. It will be appreciated that the precursor cell may be at one stage removed from the stage of differentiation where the function of the failed or damaged cell or tissue is expressed, or it may be two or three or four or more stages removed, but in each case the precursor cell is able to differentiate into the functional cell or tissue relevant to the disease to be treated.

The tolerising cells and the therapeutic cells may be, or be derived from, allogeneic adult stem cells (also called somatic stem cells). Typically, however, the tolerising cells and the therapeutic cells are derived from (but are not) embryonic stem cells, which are allogeneic. Human embryonic stem cells are typically from supernumery embryos donated by couples who have benefited from successful in vitro fertilisation (IVF) cycles and have frozen embryos that are not required in the context of the IVF treatment. Protocols for the derivation of human stem cells are well known in the art, some of which are described in U.S. Pat. No. 6,280,718 B1, incorporated herein by reference.

Derivatives of human embryonic stem cells (eg those which are lineage-specific stem cells) are functionally and physiologically similar, and sometimes identical to, somatic stem cells which all humans have and which provide us with a limited ability to repair and regenerate certain tissues. These include:
(i) Hematopoietic stem cells that give rise to all the types of blood cells: red blood cells, B lymphocytes, T lymphocytes, natural killer cells, neutrophils, basophils, eosinophils, monocytes, macrophages, and platelets.
(ii) Bone marrow stromal cells (mesenchymal stem cells) that give rise to a variety of cell types; bone cells (osteocytes), cartilage cells (chondrocytes), fat cells (adipocytes), and other kinds of connective tissue cells such as those in tendons.
(iii) Neural stem cells in the brain that give rise to its three major cell types: nerve cells (neurons) and two categories of non-neuronal cells—astrocytes and oligodendrocytes.
(iv) Epithelial stem cells in the lining of the digestive tract occur in deep crypts and that give rise to several cell types: absorptive cells, goblet cells, Paneth cells, and enteroendocrine cells.
(v) Skin stem cells occur in the basal layer of the epidermis and at the base of hair follicles. These epidermal stem cells give rise to keratinocytes, which migrate to the surface of the skin and form a protective layer. The follicular stem cells can give rise to both the hair follicle and to the epidermis.

Methods of differentiating pluripotent embryonic stem cells in to lineage-specific stem cells are known in the art. For example, U.S. Pat. No. 6,280,718 B1 to Kaufman and Thomson, incorporated herein by reference, describes a method of obtaining human haematopoietic stem cells from a culture of human pluripotent embryonic stem cells. U.S. Pat. No. 6, 458,589 B1 to Rambhatla and Carpenter, herein incorporated by reference, describes methods for producing hepatocyte lineage cells from pluripotent stem cells. Pfendler & Kawase (2003) Obstetrical & Gynecological Survey 58, 197-208 review other methods of differentiating embryonic stem cells into dopamine-producing neurons, myelin-producing oligodendrocytes, insulin-producing cells, cardiomyocytes and so on.

Although it is preferred if the cell type to be transplanted itself or a precursor thereof is used as the tolerising cell, an antigen found thereon may also be used, particularly if the antigen is a major antigen leading to histoincompatibility. Thus, MHC molecules which match the MHC antigens on the therapeutic cell may be used for tolerisation. The MHC molecules may, for instance, be used on a suitable synthetic molecular scaffold.

It will be appreciated that it is convenient in the practice of the invention for a “master” embryonic stem cell to be kept from which it is possible to produce suitable tolerising cells and therapeutic cells. It may be particularly convenient to use the earliest cell derived from an embryonic stem cell but which is committed to a particular path of differentiation (to the cell or tissue to be repaired or replaced) as the tolerising cell, and a later cell from the same differentiation pathway as the therapeutic cell. In this way, while the patient is being tolerised (using the earlier cell), cells suitable as the therapeutic cells are being produced (both derived from the same master embryonic stem cell and having common antigenic characteristics).

It will be appreciated that the tolerising cell or the therapeutic cell or both may be natural cells or may be genetically engineered cells. The tolerising cells, for example, may be genetically engineered to be more immunogenic than natural cells and so be more efficient at tolerisation, for example by overexpression of MHC antigens. The therapeutic cells may be genetically engineered to enhance their therapeutic properties, for example, cells which are able to regenerate islets of Langerhans may be genetically engineered to better produce insulin.

It is appreciated that to induce tolerance to an antigen, a derivative of the antigen may be administered to the patient, and not the antigen itself. By “derivative” of an antigen we include any portion of the antigen which can be presented by a class I or a class II MHC molecule for example on an antigen presenting cell (APC), and which induces tolerance to the antigen itself. For example, a suitable portion of an antigen is a proteolytic digest of an MHC Class II molecule from the donor. Typically the derivative of the antigen is also recognised by a T cell when presented, for example via a T cell receptor.

When the antigen is a protein, a derivative of the antigen is typically a peptide fragment of the antigen consisting of a contiguous sequence of amino acids of the antigen capable of MHC binding. Preferably, the fragment is between 6 and 100 amino acids in length. More preferably, the fragment is between 6 and 50 amino acids in length. Most preferably, the fragment is six, or seven, or eight, or nine, or ten, or eleven, or twelve, or thirteen, or fourteen, or fifteen, or sixteen, or seventeen, or eighteen, or nineteen, or twenty, or twenty-one, or twenty-two, or twenty-three, or twenty-four or twenty-five amino acids in length.

A derivative of the antigen may include a fusion of the antigen, or a fusion of a fragment of the antigen, to another compound, and which can be recognised by either a class I or a class II MHC molecule when presented, and which induces tolerance to the antigen itself. Typically, the fusion is one which can be processed by an APC so as to present a portion which is able to induce tolerance to the antigen itself.

Unless the context indicates otherwise, wherever the term “antigen” is used in the context of an antigen, a derivative as herein defined is included.

The agent which raises the effective cAMP concentration in a monocyte cell may do so in several distinct but related biochemical ways. Thus, the agent may be one which increases the production of cAMP, for example by the stimulation of receptors which are linked to the production of cAMP. Such agents include prostaglandins and agonists thereof which are described in more detail below. Cholera toxin can also be used to increase cAMP levels intracellularly as has been described in Braun et al (1999) J. Exp. Med. 189, 541-552 and there is also evidence that it may increase antigen transport across the epithelium which may be desirable. Similarly, β-adrenergic agents, which raise cAMP levels within a cell via the β-adrenergic receptor, may be used. Such β-adrenergic agents are well known in the art, such as in the treatment of asthma. Suitable β-adrenergic agents include isoproterenol.

The agent may be one which inhibits the breakdown of cAMP and thus may be a cAMP phosphodiesterase inhibitor, which are described in more detail below. The agent may be one which inhibits the export of cAMP from the cell. Export of cAMP from the cell is via a specific transporter (typically the multidrug resistance protein, MRP-4) which may be blocked with, for example, probenecid (a drug currently used for gout) or progesterone or agonists or antagonists thereof, such as medroxyprogesterone acetate or RU 486, which also appears to have an inhibitory effect on the cAMP transporter.

The agent may also be a compound which mimics the effects of cAMP in the cell in relation to generating a pro-tolerant state but which may be less susceptible to degradation or export. Such compounds, when present in the cell can be considered to raise the effective cAMP concentration. Such compounds include Sp-adenosine 3′,5′-cyclic monophosphorothioate and 8-bromoadenosine 3′,5′-cyclic monophosphate and dibutyryl cAMP. That sufficient of these compounds have been administered may be assessed by determining that there has been an elevation in IL-10 expression in monocyte cells. Preferably, the agent when used at a concentration which gives a maximal response elevates IL-10 expression at least 1.2-fold, or 1.5-fold, or 2-fold, or 5-fold, or 10-fold. Typically, from around 1 to 100 μmol of the cAMP analogues may be administered to the patient.

Forskolin is 7β-Acetoxy-8,13-epoxy-1α,6β,9α-trihydroxylabd-14-en-11-one 7β-Acetoxy-1α,6β,9α-trihydroxy-8,13-epoxy-labd-14-en-1-one. It is also called Coleonol and Colforsin and has a Mᵣ of 410. It is a cell-permeable diterpenoid that possesses anti-hypertensive, positive inotropic and adenyl cyclase activating properties. Many of its biological effects are due to its activation of adenylate cyclase and the resulting increase in intracellular cAMP concentration. Forskolin affects calcium currents and inhibits MAP kinase. Colforsin is used as daropate (see Ann Thoracic Surgery (2001) 71, 1931-1938). It may be administered as the hydrochloride to ensure water solubility but it may also be used as the free base which may be able to more readily penetrate cell membranes.

Sp-Adenosine 3′,5′-cyclic monophosphorothioate (SpcAMP) has a Mᵣ of 446 and is the Sp-diastereomer of adenosine-3′,5′-cyclic monophosphothioate. It is a potent, membrane-permeable activator of cAMP dependent protein kinase I and II that mimics the effects of cAMP as a second messenger in numerous systems while being resistant to cyclic nucleotide phosphodiesterases. It exhibits greater specificity and affinity than forskolin and cAMP analogues such as dibutyryl-cAMP.

8-Bromoadenosine 3′,5′-cyclic monophosphate (8-BrcAMP) has a Mᵣ of 430. It is a cell-permeable cAMP analogue having greater resistance to hydrolysis by phosphodiesterases than cAMP. It activates protein kinase A.

Cholera toxin has a Mᵣ of around 100,000. It is a toxin consisting of an A subunit (27 kDa) surrounded by five B subunits (approximately 12 ka each), which attach the toxin to ganglioside GM1 on the cell surface. The A subunit catalyzes ADP-ribosylation of the α-subunit of the stimulatory G protein (Gas) reducing GTPase activity and activating the α-subunit. This activation of Gas leads to an increase in the activity of adenylate cyclase resulting in increased levels of cAMP. It also ADP-ribosylates transducin in the eye rod outer segments, inactivating its GTPase activity. Cholera toxin has also been reported to ADP-ribosylate tubulin. It has been shown to be a potent mucosal vaccine adjuvant, inducing T helper cell type 2 responses by inhibiting the production of interleukin-12 (Braun et al (1999) supra). Although fragments of cholera toxin which are able to increase cAMP levels in monocytes may be used, it is preferred that complete cholera toxin is used.

Since cholera toxin may, under some conditions, induce anaphylaxis (oversensitization), it is less preferred.

It is likely that SpcAMP and 8-BrcAMP, agents such as rolipram and possibly forskolin, inhibit the cA export pump and this may contribute to their ability for raising the effective cAMP concentration.

It is convenient to measure the effective cAMP concentration in monocyte cells (ie by assessing the effect of the agent on monocyte cells). A preferred monocyte cell is the well known human monocyte cell line U937. It will be appreciated that the agents will also raise the effective cAMP concentration in other monocyte and monocyte-related cells such as macrophages, and that the utility in the context of the invention may be due to the effect on these cells. As noted above, whether or not there is a sufficient amount of cAMP analogues can be determined by measuring IL-10 in monocyte cells. Preferably, the agent when used at a concentration which gives a maximal response raises the cAMP concentration at least 1.2-fold, or 1.5-fold, or 2-fold, or 5-fold, or 10-fold.

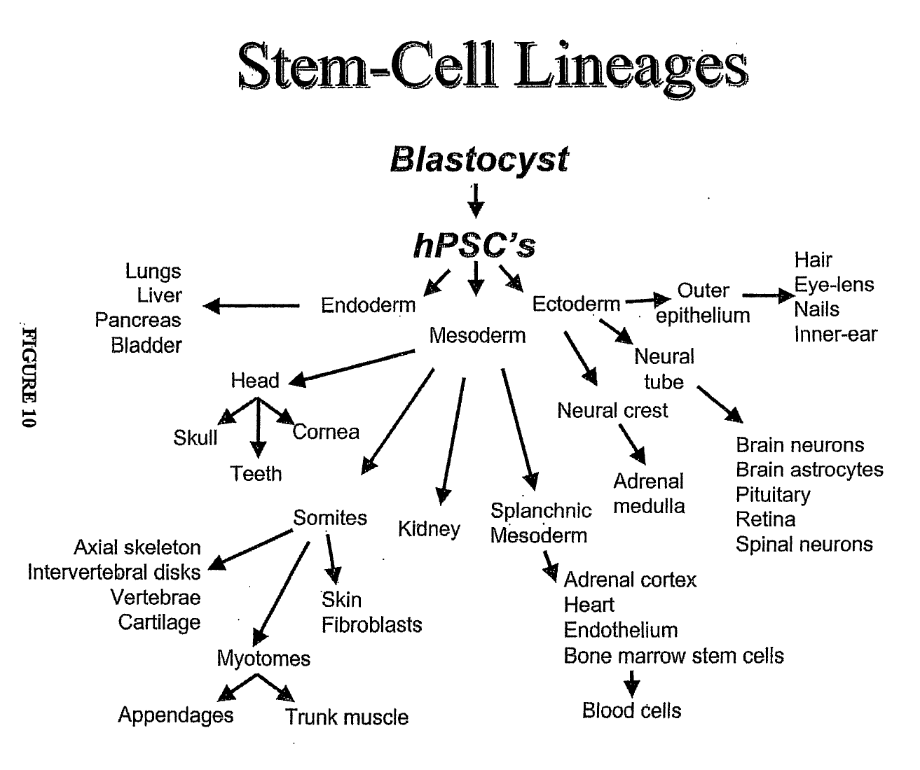

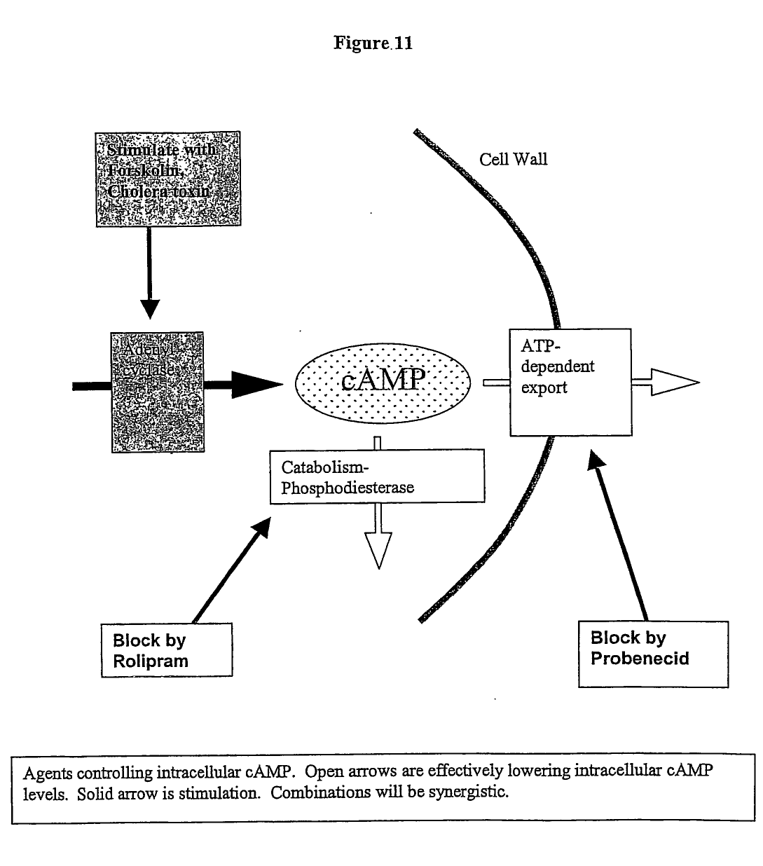

FIG. 11 shows diagrammatically various places of intervention in or on a cell which lead to raising cAMP levels.

It is preferred that the agent which raises the effective cAMP concentration in a monocyte cell is a prostaglandin.

It is preferred for this and all other aspects of the invention that the prostaglandin or agonist thereof stimulates cAMP production in a monocyte.

The prostaglandin or agonist thereof may be any suitable prostaglandin or agonist thereof that stimulates cAMP production in a monocyte, and which particularly in the presence of GMCSF causes monocytes to express IL-10. Prostaglandins or agonists thereof that are suitable for use in the present invention may readily be determined by a person of skill in the art. Methods for assessing cAMP production in monocytes may be found in Burzyn et al, (2000) and in Example 3, and methods for detecting IL-10 expression in and release from monocytes include those in Examples 1 and 3.

By “prostaglandin or agonist” we mean any compound which acts as a prostaglandin agonist on a prostaglandin receptor. The prostaglandin agonist may be, but need not be, a prostanoid. Typically, the prostaglandin or agonist is one which binds the EP2 or EP4 receptor. The prostaglandin may be a PGE, a PGD or a PGI, or an agonist thereof. Preferably, the prostaglandin is a PGE or an agonist thereof. It is appreciated that PGI may be too unstable to be useful as a pharmacological agent, however PGI₂ and stable analogues of PGI may be suitable. Preferably, the prostaglandin is not a PGF or an agonist thereof.

It is preferred that the prostaglandin or agonist thereof is PGE₂ or a synthetic analogue thereof. Synthetic analogues include those modified at position 15 or 16 by the addition of a methyl group or those where the hydroxyl has been transposed from position 15 to position 16. Preferred examples of analogues of prostaglandin include Butaprost (an EP2 receptor agonist) and 11-deoxy PGE1 (an EP4 receptor agonist) and 19-hydroxy PGE. For the avoidance of doubt, the term “prostaglandin” includes naturally-occurring prostaglandins as well as synthetic prostaglandin analogues.

Suitable prostaglandins or agonists thereof include dinoprostone (sold as Propess by Ferring in Europe and Forest in the USA; sold as Prostin E2 by Pharmacia), gemeprost (sold by Farillon), misoprostol (which is sold as Cytotec by Searle and Pharmacia), alprostadil (which is sold as Caverect by Pharmacia and Viridal by Schwarz and MUSE by AstraZeneca) and limaprost.

Misoprostol is a PGE analogue which has EP2 and EP3 agonist effects. Its chemical structure is (±) methyl 11α, 16-dihydroxy-16-methyl-9-oxoprost-13-enoate.

An example of a non-prostanoid compound which acts as a prostaglandin agonist is AH23848, an EP4 receptor agonist.

EP2 agonists which may be usefull in the practise of the invention include AH13205.

Suitable prostaglandins also include 19-hydroxy PGE1 and 19-hydroxy PGE2. Prostaglandin E agonists are described in EP 1 097 922 and EP 1 114 816, incorporated herein by reference.

Suitable prostaglandins or agonists thereof may also include any of the 19-hydroxy prostaglandin analogues described in U.S. Pat. No. 4,127,612, incorporated herein by reference.

It is preferred that the prostaglandin is prostaglandin E₂ (PGE₂) or 19-hydroxy PGE. Prostaglandins and agonists thereof, including PGE₂, are commercially available, for example from Pharmacia and Upjohn as Prostin E2.

The inventors further believes that it may be beneficial to use a phosphodiesterase (PDE) inhibitor either alone or with other agents which raise the effective cAMP concentration in a monocyte cell. The principal receptors for prostaglandin E2 (PGE2) are the EP2 and EP4 sub-types; however, other receptor sub-types exist (namely EP1 and EP3). EP2 and EP4 receptors couple with adenylcyclase and use elevated cAMP as the messenger system. The levels of cAMP in tissue are governed both by its synthesis and by its catabolism by PDEs which can be blocked by specific PDE inhibitors. Thus, the inventor believes that the effect of a prostaglandin or agonist thereof (such as PGE) acting on its EP2 and EP4 receptors is to stimulate cAMP, and the addition of the PDE inhibitor provides a synergistic action on monocytes and macrophages resulting in a reduction in the immune and/or inflammatory response which is greater than the effect of the sum of the same amount of the prostaglandin or agonist thereof, or PDE inhibitor administered alone.

Moreover, the inventors have previously found that the combination of a prostaglandin and a PDE inhibitor markedly stimulate IL-10 and inhibit IL-12 expression in, and secretion from, cells of the immune system, resulting in a tolerising environment.

Thus in an embodiment, the composition may further comprise a PDE inhibitor.

The PDE inhibitor may be any suitable PDE inhibitor. Preferably, the PDE inhibitor is one which inhibits a PDE which is active in cAMP breakdown. The PDEs which are known to be active in cAMP breakdown are those of the types IV, VII and VIII. Preferably, the PDE inhibitors are selective for type IV or VII or VIII.

Most preferably, the PDE inhibitors are selective for type IV PDE. By “selective” we mean that the inhibitor inhibits the particular type of PDE inhibitor for which it is selective, more potently than another type. Preferably, the type IV selective inhibitor is at least 2 times more potent an inhibitor of type IV PDE than another PDE type. More preferably, the type IV selective inhibitor is at least 5 times, 10 times, 20 times, 30, times 40 times, 50 times, 100 times, 200 times, 500 times or 1000 times more potent an inhibitor of type IV PDE than another PDE type.

Typically, the selective inhibitor is around 5 to 50 times more potent an inhibitor of the selected PDE type than another PDE type. Typically, the selective inhibitor is 5 to 50 times more potent an inhibitor of the selected PDE type than an inhibitor that is considered to be non-selective such as theophylline. Thus, theophylline is 30 times less effective than rolipram.

Preferably, selective inhibition is determined by a comparison of IC₅₀ levels (Dousa (1999) Kidney International 55: 29-62).

Non-specific PDE inhibitors include caffeine, theophylline, 3-isobutyl-1-methylxanthine (IBMX) and pentoxifylline (3,7-dihydro-3,7-dimethyl-1-(5-oxohexyl)-1H-purine-2,6-dione), although caffeine is not as active as the others and so is less preferred The IC₅₀ value for IBMX is 2-50 μM.

U.S. Pat. No. 6,127,378, incorporated herein by reference, discloses phenanthridines substituted in the 6 position that are described as selective PDE inhibitors (mainly of type IV), that may be suitable for use in the methods of the invention.

Specific (or selective) type IV PDE inhibitors include -rolipram (4-[3-cyclopentyloxy-4-methoxyphenyl]-2-pyrrolidinone) and Ro-20-1724 (4-[3-butoxy-4-methoxybenzyl]-2-imidazolidinone). The IC₅₀ for rolipram is 800 nM, and the IC₅₀ for Ro-20-1724 is 2 μM.

Another suitable PDE type IV selective inhibitor is denbufylline (1,3-di-n-butyl-7-(2-oxopropyl)-xanthine).

CP 80 633 (Hamfin et al (1996) J. Invest. Dermatol. 107, 51-56), CP 102 995 and CP 76 593 are also all potent type IV inhibitors (available from Central Research Division, Pfizer Inc, Groton, Conn.).

Other high affinity type IV selective PDE inhibitors include CPD 840, RP 73401, and RS 33793 (Dousa, 1999). The high affinity type IV selective PDE inhibitors have a Kᵢ of approximately 1 nM while the lower affinity inhibitors have a Kᵢ of about 1 μM.

The disclosures in Dousa (1999); Müller et al (1996, Trends Pharmacol. Sci. 17: 294-298); Palfreyman & Souness (1996, Prog Med Chem 33: 1-52); Stafford & Feldman (1996, Annual Reports in Medicinal Chemistry (vol 31) pp 71-80; Ed. Bristol, Academic Press, NY, USA); and Teixeira et al (1997, Trends Pharmacol. Sci. 18: 164-171) relating to type IV PDE selective inhibitors are incorporated herein by reference.

Typically, when a type IV PDE-selective inhibitor is administered orally, around 1 to 30 mg is used. Thus, a typical oral dose of rolipram or denbufylline is 1 mg or 5 mg or 10 mg or 30 mg. When a non-selective PDE inhibitor is used, such as theophylline, and it is administered orally, the dose is between 5 and 50 mg, such as 5 or 10 or 20 or 30 or 40 or 50 mg.

When the composition includes progesterone, it is preferred if the dose of progesterone is sufficient to provide levels of between 100 nM and 50 μM.

Preferred combinations are:
PGE
PGE+Rolipram
PGE+probenecid
PGE+Rolipram+probenecid
Forskolin
Forskolin+Rolipram
Forslkolin+Rolipram+probenecid
8-Bromo cAMP+probenecid
8-Bromo cAMP+Rolipram+probenecid
Sp-Adenosine 3,5-cyclic monophosphothioate (SpcAMP)
SpcAMN+probenecid
SpcAMP+Rolipram+probenecid
Cholera toxin
Cholera toxin+probenecid

Preferably, these (and other agents which raise the effective cAMP concentration in a monocyte cell) are combined with GMCSF.

The inventors believe that these (and.other) combinations may act synergistically to desirably raise the effect cAMP levels in monocyte cells. It will also be appreciated that by manipulating all the metabolic points for cAMP (see FIG. 11), a lower dose of the components of the mixture would be possible in order to give the same effect compared to a single component alone.

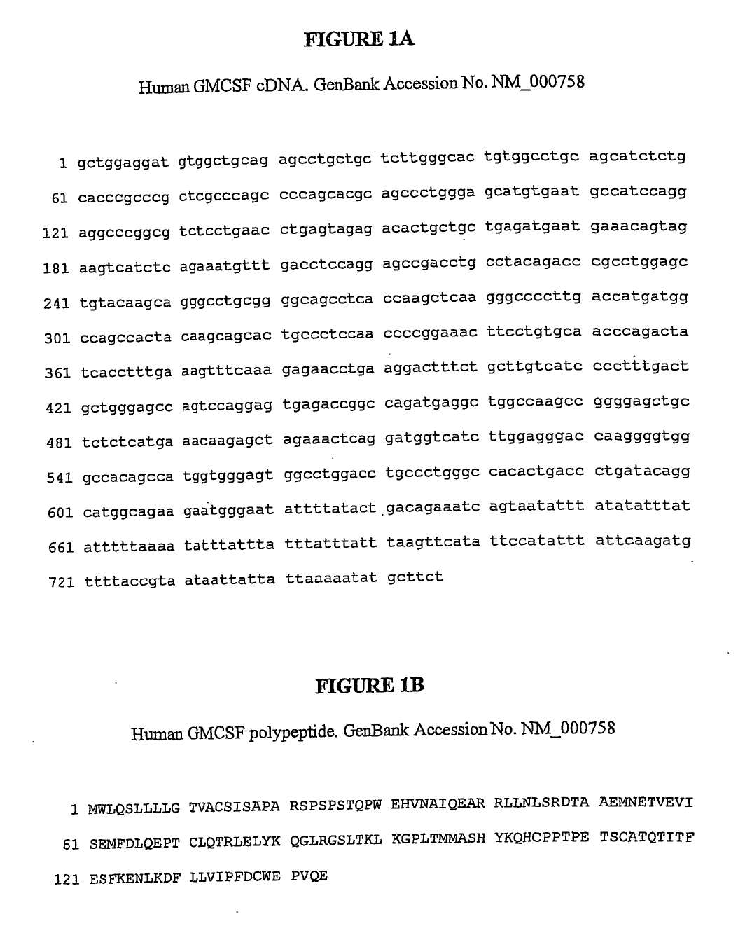

By “GMCSF” we include the gene product of the human GMCSF gene and naturally occurring variants thereof. The nucleotide and the amino acid sequence of human GMCSF is found in Genbank Accession No. NM_{—}000758, and in FIG. 1. Some naturally occurring variants of GMCSF are also listed in NM_{—}000758. GMCSF is also known as colony stimulating factor 2 (CSF2).

The invention includes the use of derivatives of GMCSF that retain the biological activity of wild-type GMCSF, ie that stimulate the production of granulocytes and macrophages from their progenitor cells, and which in the presence of prostaglandin E cause monocytes to express IL-10.

By “derivative” of GMCSF we include a fragment, fusion or modification or analogue thereof, or a fusion or modification of a fragment thereof.

By “fragment” of GMCSF we mean any portion of the glycoprotein that stimulates the production of granulocytes and macrophages from their progenitor cells and which in the presence of prostaglandin E causes monocytes to express IL-10. Typically, the fragment has at least 30% of the activity of full length GMCSF. It is more preferred if the fragment has at least 50%, preferably at least 70% and more preferably at least 90% of the activity of full length GMCSF. Most preferably, the fragment has 100% or more of the activity of full length GMCSF.

The derivatives may be made using protein chemistry techniques for example using partial proteolysis (either exolytically or endolytically), or by de novo synthesis. Alternatively, the derivatives may be made by recombinant DNA technology. Suitable techniques for cloning, manipulation, modification and expression of nucleic acids, and purification of expressed proteins, are well known in the art and are described for example in Sambrook et al (2001) “Molecular Cloning, a Laboratory Manual”, 3^{rd} edition, Sambrook et al (eds), Cold Spring Harbor Laboratory Press, Cold Spring Harbor, NY, USA, incorporated herein by reference.

The invention also includes modifications of full length GMCSF, or a fragment thereof, that stimulate the production of granulocytes and macrophages from their progenitor cells and which in the presence of prostaglandin E cause monocytes to express IL-10.

Such modifications include deglycosylating the glycoprotein either fully or partially. Other modifications include full length GMCSF, or a fragment thereof, having a different glycosylation pattern from that found in naturally occurring human GMCSF.

Other modifications of full length GMCSF, or a fragment thereof, include amino acid insertions, deletions and substitutions, either conservative or non-conservative, at one or more positions. Such modifications may be called analogues of GMCSF. By “conservative substitutions” is intended combinations such as Gly, Ala; Val, Ile, Leu; Asp, Glu; Asn, Gin; Ser, Thr; Lys, Arg; and Phe, Tyr. Such modifications may be made using the methods of protein engineering and site-directed mutagenesis, as described in Sambrook et al 2001, supra. Preferably, the modified GMCSF or modified GMCSF fragment retains at least 30% of the activity of full length GMCSF. It is more preferred if the modified GMCSF or GMCSF derivative has at least 50%, preferably at least 70% and more preferably at least 90% of the activity of fill length GMCSF. Most preferably, the modified GMCSF or modified GMCSF fragment has 100% or more of the activity of full length GMCSF.

The invention also includes the use of a fusion of fill length GMCSF, or a fragment thereof, to another compound. Preferably, the fusion retains at least 30% of the activity of fall length GMCSF. It is more preferred if the fusion has at least 50%, preferably at least 70% and more preferably at least 90% of the activity of full length GMCSF. Most preferably, the fusion has 100% or more of the activity of full length GMCSF.

GMCSF and analogues thereof are described in the following publications, each of which are incorporated herein by reference: U.S. Pat. No. 5,229,496 (Deeley et al.); U.S. Pat. No. 5,391,485 (Deeley et al.); U.S. Pat. No. 5,393,870 (Deeley et al.); U.S. Pat. No. 5,602,007 (Dunn et al); Wong et al, “Human GM-CSF: molecular cloning of the complementary DNA and purification of the natural and recombinant proteins”, Science 228 (4701), 810-815 (1985); Lee et al, “Isolation of cDNA for a human granulocyte-macrophage colony-stimulating factor by functional expression in mammalian cells”, Proc. Natl. Acad Sci. U.S.A. 82 (13), 4360-4364 (1985); Cantrell et al, “Cloning, sequence, and expression of a human granulocyte/macrophage colony-stimulating factor”, Proc. Natl. Acad. Sci. U.S.A. 82 (18), 6250-6254 (1985); and Miyatake et al, “Structure of the chromosomal gene for granulocyte-macrophage colony stimulating factor: comparison of the mouse and human genes”, EMBO J. 4 (10),2561-2568 (1985).

While it is preferred that GMCSF is human GMCSF as defined above, by GMCSF we also include GMCSF from other species. However, it is appreciated that for applications in which GMCSF is administered to a subject, the GMCSF is preferably from the same species as the subject. Thus if the GMCSF is to be administered to a human subject, the GMCSF is preferably human GMCSF.

Suitable GMCSF for the practice of this invention can be obtained from Peprotech EC Ltd., 29 Margravine Road, London, W6 8LL, catalogue number 300-03.

A preferred GMCSF for the practice of this invention is sargramostim, the proper name for yeast-derived recombinant human GMCSF, sold under the trade name Leukine® produced by Immunex, Inc. Leukine® is a recombinant human GMCSF produced in a S. cerevisiae expression system. Leukine® is a glycoprotein of 127 amino acids characterised by 3 primary molecular species having molecular masses of 19,500, 16,800 and 15,500 Daltons. The amino acid sequence of Leukine® differs from natural human GMCSF by a substitution of leucine at position 23, and the carbohydrate moiety may be different from the native protein. Leukine® is suitable for subcutaneous or intravenous administration (Leukine® Package Insert Approved Text, February 1998).

Unless the context indicates otherwise, wherever the term “GMCSF” is used, a derivative as herein defined is included.

In an embodiment, a monocyte-attracting chemotactic agent may also be used in aiding the production of a tolerising environment by attracting monocytes.

Suitable chemotactic agents for the practice of this invention include MIP-1α and MCP-1, which can be obtained from Peprotech EC Ltd., 29 Margravine Road, London, W6 8LL, catalogue number 300-04. Other suitable chemotactic agents are described in U.S. Pat. No. 5,908,829 to Kelly, incorporated herein by reference.

Typically in the first, second and third aspects of the invention, the tolerising cell or an antigen found thereon or a derivative of said antigen and the agent which raises the effective cAMP concentration in a monocyte cell are administered simultaneously to the patient. More typically, they are all present in the same composition (such as a pharmaceutical composition or formulation; see below). However, it is possible for the components to be administered separately, in which case it is desirable that the agent which raises the effective cAMP concentration in a monocyte cell are administered prior to administration of the tolerising cell or an antigen found thereon or a derivative of said antigen. Typically, if there is a time lag between administering the agent and the cells, it will be of the order of minutes.

Typically, in the third aspect of the invention, the therapeutic amount of the therapeutic cells are administered after tolerance has been achieved by the administration of the tolerising cells and the said agent. Thus, the patient is pretolerised to the cells prior to the therapeutic administration of therapeutic cells. Typically, the time between the pretolerisation regime and the therapeutic administration of therapeutic cells is of the order of 1 to 10 days.

The administration of the tolerising cells and the said agent typically is to a convenient site where the components can interact with the immune system and give rise to the induction of tolerance. Conveniently, a “tolerising” complex of the cells and the agent is used and administered to a mucous membrane which can be accessed non-invasively. Thus, suitable mucous membranes include those found in the mouth, vagina, anus, gastrointestinal tract and nose. Typically, therefore, the components are formulated as a buccal tablet, as a pessary, vaginal tablet or ring, or as a suppository or as a nasal spray.

Administration of the therapeutic amount of the precursor cell is directly or indirectly to the site where it is required in order regenerate failed or damaged cells or tissues. Typically, this is to the site of degeneration or damage or trauma and will vary depending on the disease or disorder to be treated.

The number of tolerising cells used may vary but would typically be around 100 to 10⁶ cells. Sufficient of the agent which raises effective cAMP concentration in a monocyte cell is administered in order to produce a tolerising environment in the patient Typically, around 2 μmol of prostaglandin may be administered, around 50-100 ng GMCSF and around 10 μmol of a PDE inhibitor. When combinations are used, it is envisaged that lower amount of individual components will be required.

Sufficient therapeutic cells are administered to give a beneficial effect, such as initiation of repair or regeneration of the diseased or damaged cells or tissues. Typically around 10⁵ to 10⁸ therapeutic cells are administered, such as 10⁶ or 10⁷ cells.

How the cells are introduced to the site of disease or trauma varies. For example, for the treatment of diabetes, the “Edmonton protocol” may be used in which islet cells or immediate precursors thereof are injected into the portal vein of the liver in which organ they form effective, physiologically normal, glucose responsive and insulin-producing islets (see http://www.diabetes.org.uk/islets/trans/edmonton.htm for more details of the protocol). For the treatment of Parkinson's disease, cells may be injected into one of the accessible ventricles or portal veins from where they migrate to the correct site in the substantia nigra. For cardiac indications, and spinal chord injuries, cells may be injected directly to the site of damage to be repaired.

A further aspect of the invention provides a composition for inducing tolerance to a therapeutic cell in a patient who is to be administered subsequently a therapeutic amount of the said therapeutic cell or a precursor thereof comprising (a) a tolerising cell sharing the same antigenic characteristics as the therapeutic cell, or an antigen found thereon or a derivative of said antigen, (b) an agent which raises the effective cAMP concentration in a monocyte cell, optionally, (c) granulocyte-macrophage colony stimulating factor (GMCSF) or a derivative thereof As noted above, such a composition is useful for inducing tolerance. Conveniently, the composition is packaged and presented for use as a medicament, including as a medicament for human or veterinary use. Typically, the composition is packaged and presented for use in inducing tolerance to the therapeutic cell.

A still further aspect of the invention provides a therapeutic system for inducing tolerance to a therapeutic cell in a patient who is to be administered subsequently a therapeutic amount of the said therapeutic cell or a precursor thereof comprising (a) a tolerising cell sharing the same antigenic characteristics as the therapeutic cell, or an antigen found thereon or a derivative of said antigen, (b) an agent which raises the effective cAMP concentration in a monocyte cell, optionally, (c) granulocyte-macrophage colony stimulating factor(GMCSF) or a derivative thereof.

A yet still further aspect of the invention provides a kit of parts for inducing tolerance to a therapeutic cell in a patient who is to be administered subsequently a therapeutic amount of the said therapeutic cell or a precursor thereof comprising (a) a tolerising cell sharing the same antigenic characteristics as the therapeutic cell, or an antigen found thereon or a derivative of said antigen, (b) an agent which raises the effective cAMP concentration in a monocyte cell, optionally, (c) granulocyte-macrophage colony stimulating factor (GMCSF) or a derivative thereof

The therapeutic system and kit of parts again are useful for inducing tolerance in a patient to the therapeutic cell. Optionally, the therapeutic system or kit of parts may additionally contain a therapeutic cell which is, or is able to differentiate into, the cell or tissue to be regenerated.

A pharmaceutical composition comprising a composition for inducing tolerance to a therapeutic cell in a patient who is to be administered subsequently a therapeutic amount of the said therapeutic cell or a precursor thereof comprising (a) a tolerising cell sharing the same antigenic characteristics as the therapeutic cell, or an antigen found thereon or a derivative of said antigen, (b) an agent which raises the effective cAMP concentration in a monocyte cell, optionally, (c) granulocyte-macrophage colony stimulating factor (GMCSF) or a derivative thereof, and a pharmaceutically acceptable carrier is also included in the invention

The carrier, diluent or excipient must be “acceptable” in the sense of being compatible with the composition of the invention and not deleterious to the recipients thereof. Typically, the carriers will be water or saline which will be sterile and pyrogen free. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), or solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Further aspects of the invention include the following:

Use of a combination of (a) a tolerising cell sharing the same antigenic characteristics as the therapeutic cell to be administered therapeutically, or an antigen found thereon or a derivative of said antigen and (b) an agent which raises the effective cAMP concentration in a monocyte cell, in the manufacture of a medicament for inducing tolerance to a therapeutic cell in a patient who is to be administered subsequently a therapeutic amount of the said therapeutic cell or a precursor thereof.

Use of a tolerising cell sharing the same antigenic characteristics as a therapeutic cell to be administered therapeutically, or an antigen found thereon or a derivative of said antigen in the manufacture of a medicament for inducing tolerance to a therapeutic cell in a patient who is to be administered subsequently a therapeutic amount of the said therapeutic cell wherein the patient is administered an agent which raises the effective cAMP concentration in a monocyte cell.

Use of an agent which raises the effective cAMP concentration in a monocyte cell in the manufacture of a medicament for inducing tolerance to a therapeutic cell in a patient who is to be administered subsequently a therapeutic amount of the said therapeutic cell wherein the patient is administered a tolerising cell sharing the same antigenic characteristics as the therapeutic cell to be administered therapeutically, or an antigen found thereon or a derivative of said antigen.

Use of any one or two of (a) a tolerising cell sharing the same antigenic characteristics as the therapeutic cell to be administered therapeutically, or an antigen found thereon or a derivative of said antigen, (b) an agent which raises the effective cAMP concentration in a monocyte cell and (c) GMCSF, in the manufacture of a medicament for inducing tolerance to the therapeutic cell in a patient who is to be administered subsequently a therapeutic amount of the therapeutic cell, and who is administered one or both of (a), (b) or (c) which is not found in the medicament as said.

Use of a combination of (a) a tolerising cell sharing the same antigenic characteristics as a therapeutic cell which therapeutic cell is, or is able to differentiate into, the cell or tissue to be regenerated, or an antigen found thereon or a derivative of said antigen, and (b) an agent which raises the effective cAMP concentration in a monocyte cell, in the manufacture of a medicament for reducing the risk of rejection of a transplant in a patient in need of transplantation of a therapeutic cell for cell or tissue regeneration.

Use of a tolerising cell sharing the same antigenic characteristics as a therapeutic cell which therapeutic cell is, or is able to differentiate into, the cell or tissue to be regenerated, or an antigen found thereon or a derivative of said antigen in the manufacture of a medicament for reducing the risk of rejection of a transplant in a patient in need of transplantation of a therapeutic cell for cell or tissue regeneration wherein the patient is administered an agent which raises the effective cAMP concentration in a monocyte cell.

Use of an agent which raises the effective cAMP concentration in a monocyte cell in the manufacture of a medicament for reducing the risk of rejection of a transplant in a patient in need of transplantation of a therapeutic cell for cell or tissue regeneration wherein the patient is administered a tolerising cell sharing the same antigenic characteristics as the therapeutic cell which therapeutic cell is, or is able to differentiate into, the cell or tissue to be regenerated, or an antigen found thereon or a derivative of said antigen.

Use of any one or two of (a) a tolerising cell sharing the same antigenic characteristics as a therapeutic cell which therapeutic cell is, or is able to differentiate into, the cell or tissue to be regenerated, or an antigen found thereon or a derivative of said antigen, (b) an agent which raises the effective cAMP concentration in a monocyte cell and (c) GMCSF in the manufacture of a medicament for reducing the risk of rejection of a transplant in a patient in need of transplantation of a therapeutic cell for cell or tissue regeneration, and who is administered one or both of (a), (b) or (c) which is not found in the medicament as said.

Use of a combination of (a) a tolerising cell sharing the same antigenic characteristics as the therapeutic cell to be administered subsequently which therapeutic cell is, or is able to differentiate into, the cell or tissue to be regenerated, or an antigen found thereon or a derivative of said antigen, and (b) an agent which raises the effective cAMP concentration in a monocyte cell in the manufacture of a medicament for treating a patient in need of cell or tissue regeneration, wherein the patient is subsequently administered a therapeutic amount of the said therapeutic cell.

Use of a tolerising cell sharing the same antigenic characteristics as the therapeutic cell to be administered subsequently which therapeutic cell is, or is able to differentiate into, the cell or tissue to be regenerated, or an antigen found thereon or a derivative of said antigen in the manufacture of a medicament for treating a patient in need of cell or tissue regeneration wherein the patient is administered an agent which raises the effective cAMP concentration in a monocyte cell and is subsequently administered a therapeutic amount of the said therapeutic cell.

Use of an agent which raises the effective cAMP concentration in a monocyte cell in the manufacture of a medicament for treating a patient in need of cell or tissue regeneration wherein the patient is administered a tolerising cell sharing the same antigenic characteristics as the therapeutic cell to be administered subsequently which therapeutic cell is, or is able to differentiate into, the cell or tissue to be regenerated, or an antigen found thereon or a derivative of said antigen and is subsequently administered a therapeutic amount of the said therapeutic cell.

Use of any one or two of (a) a tolerising cell sharing the same antigenic characteristics as the therapeutic cell to be administered subsequently which therapeutic cell is, or is able to differentiate into, the cell or tissue to be regenerated, or an antigen found thereon or a derivative of said antigen, (b) an agent which raises the effective cAMP concentration in a monocyte cell and (c) GMCSF or a derivative thereof in the manufacture of a medicament for treating a patient in need of cell or tissue regeneration, and who is administered one or two of (a), (b) or (c) which is not found in the medicament as said, wherein the patient is subsequently administered a therapeutic amount of the said therapeutic cell.

Use of a therapeutic amount of a therapeutic cell which is, or is able to differentiate into, a cell or tissue to be regenerated in the manufacture of a medicament for treating a patient in need of cell or tissue regeneration, wherein the patient has previously been administered (a) a tolerising cell sharing the same antigenic characteristics as the therapeutic cell, or an antigen found thereon or a derivative of said antigen and (b) an agent which raises the effective cAMP concentration in a monocyte cell and, optionally, (c) GMCSF.

The invention will now be described in more detail with the aid of the following Figures and Examples.

FIG. 1

cDNA and amino acid sequence (FIGS. 1A and 1B, respectively) of human GMCSF, taken from GenBank Accession No. NM_{—}000758.

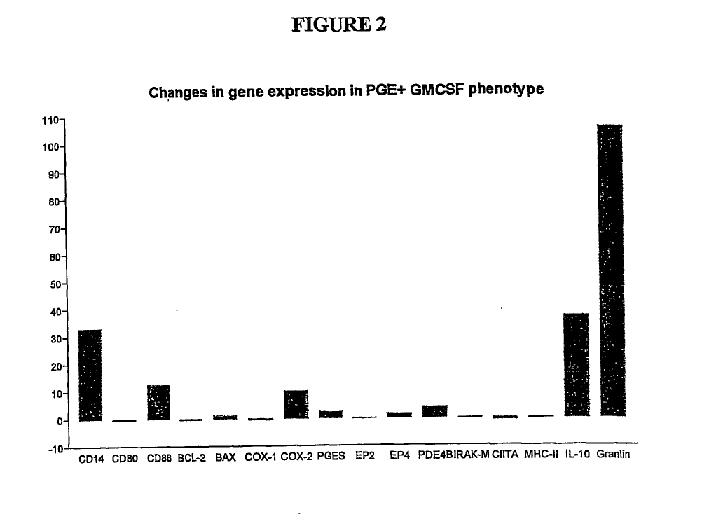

FIG. 2

FIG. 2 is a graph showing the effect of PGE and GMCSF on gene expression in U937 cells. Cells were treated for 4 hours with PGE2, with and without GMCSF, washed to remove the treatment, and incubated for a further 20 hours before the cells were pelleted and RNA extracted. The mRNA levels of CD14, CD80, CD86, BCL-2, BAX, COX-1 (cyclo-oygenase 1), COX-2, PGES (prostaglandin synthase), EP2 (a prostaglandin receptor), EP4 (a prostaglandin receptor), PDE4B (a phosphodiesterase), IRAK-M, CIITA (NHC class II transactivator), MHC-II, IL-10 and granulysin (abbreviated to granlin), were measured. The graph indicates the percentage change in expression levels in the presence of GMCSF and PGE2.

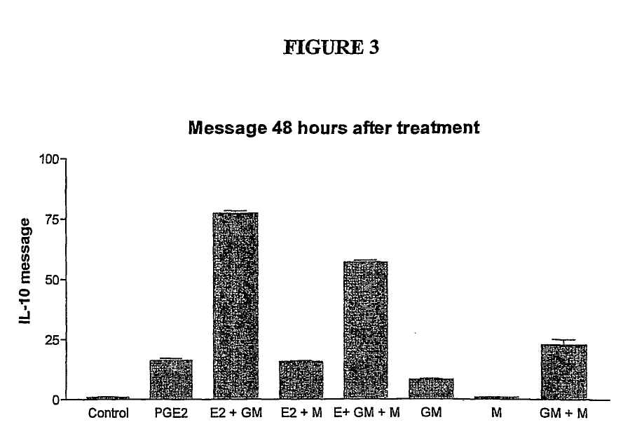

FIG. 3

FIG. 3 is a graph showing the synergistic effect of PGE and GMCSF on the production of IL-10 mRNA in U937 cells, and that this phenotype is maintained 48 hours after removal of the treatment. Cells were treated for 4 hours with the agents indicated below the graph, washed to remove the treatment, and incubated for a further 48 hours before the cells were pelleted and RNA extracted. PGE2, E2 and E all refer to prostaglandin E2; GM refers to GMCSF; and M refers to MCSF.

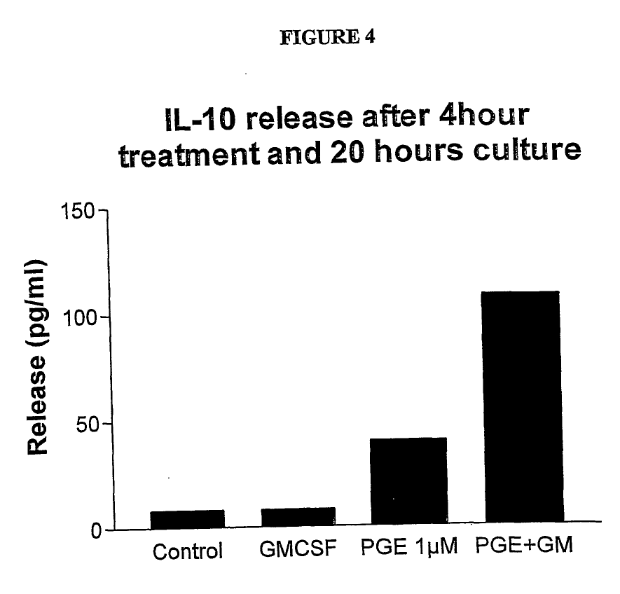

FIG. 4

FIG. 4 is a graph showing the synergistic effect of PGE and GMCSF on the release of IL-10 protein in U937 cells, and that this phenotype is maintained after removal of the treatment. Cells were treated for 4 hours with the agents indicated below the graph, washed to remove the treatment and incubated for a further 20 hours before the medium was assayed for IL-10. PGE refers to prostaglandin E2, and GM refers to GMCSF.

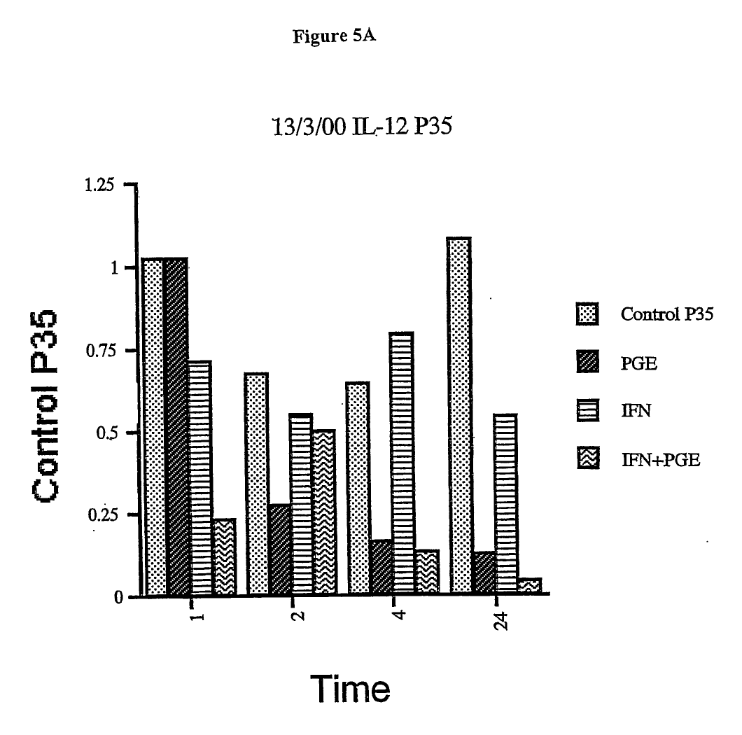

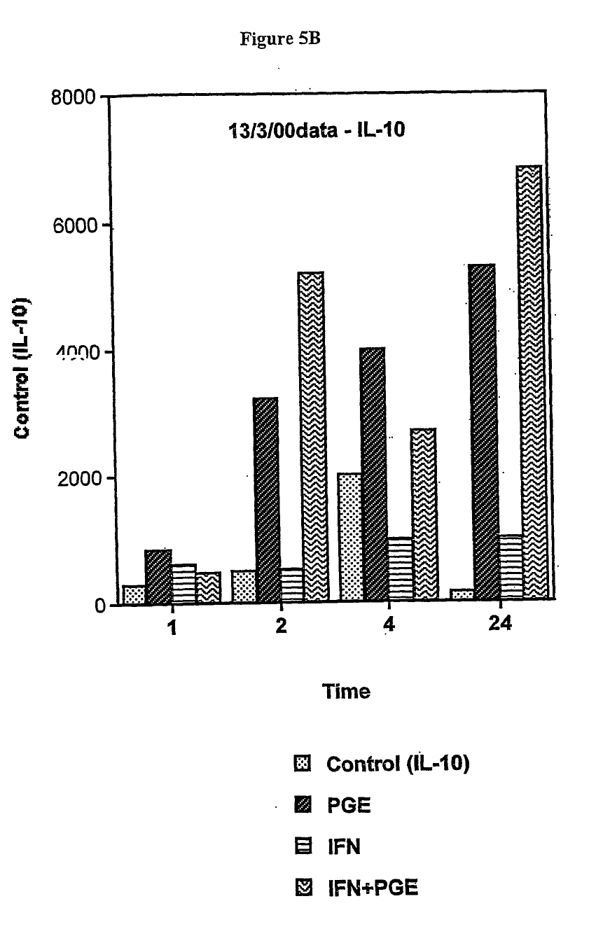

FIG. 5

Expression of mRNA for cytokines IL-10 and IL-12 subunit p35. Experiment carried out on U937 cells (pro-monocytes) in the presence of Rolipram at 1 μg/ml=4 μM and indomethacin 10 μM. The indomethacin prevents prostaglandin synthesis from cells. Note that the effect of PGE+Rolipram is a marked stimulation of IL-10 and an inhibition of IL-12 both for unstimulated and IFNγ stimulated cells. Vertical scale is a measure of mRNA compared to a control sample as measured by real-time quantitative PCR (Taqman).

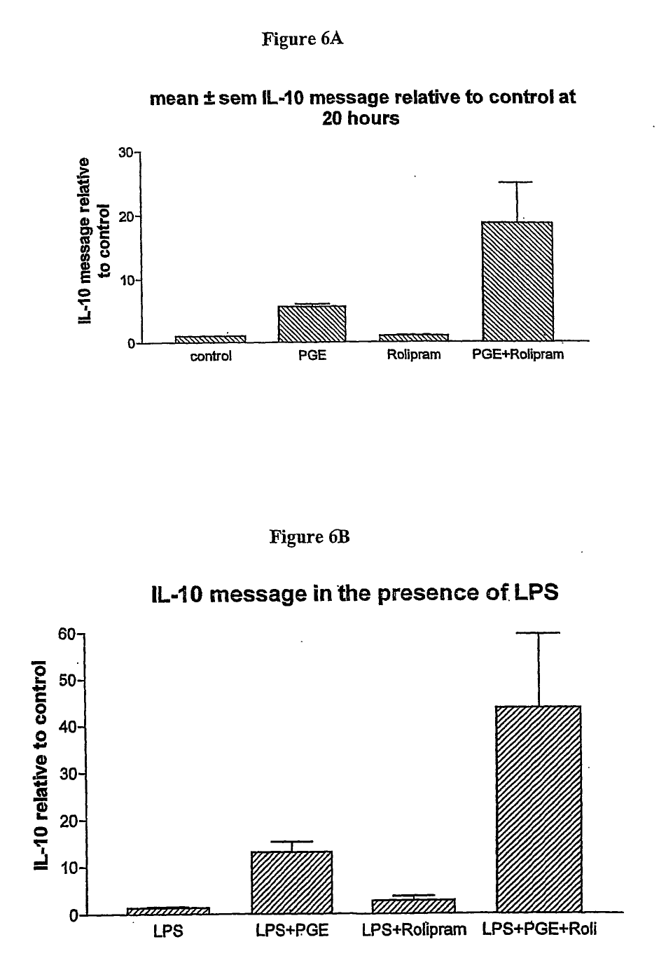

FIG. 6

FIG. 6A is a graph showing the effect of PGE and Rolipram on the production of IL-10 mRNA in U937 cells. FIG. 6B is a graph showing the effect of LPS, PGE and Rolipram on the production of IL-10 mRNA in U937 cells. FIG. 6C is a graph showing the effect of LPS, PGE and Rolipram on IL-10 release from U937 cells. FIG. 6D is a graph showing the effect of PGE and Rolipram on IL-10 release from U937 cells.

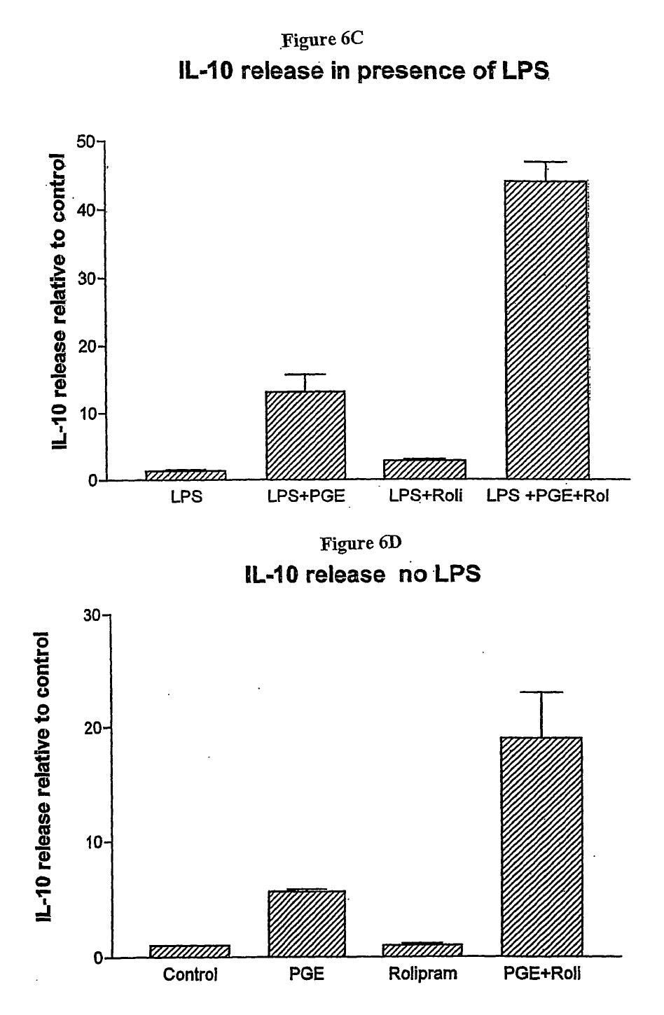

FIG. 7

A graph showing the effect of 19 hydroxy PGE1 and 19 hydroxy PGE2 on the stimulation of IL-10 in the presence and absence of rolipram.

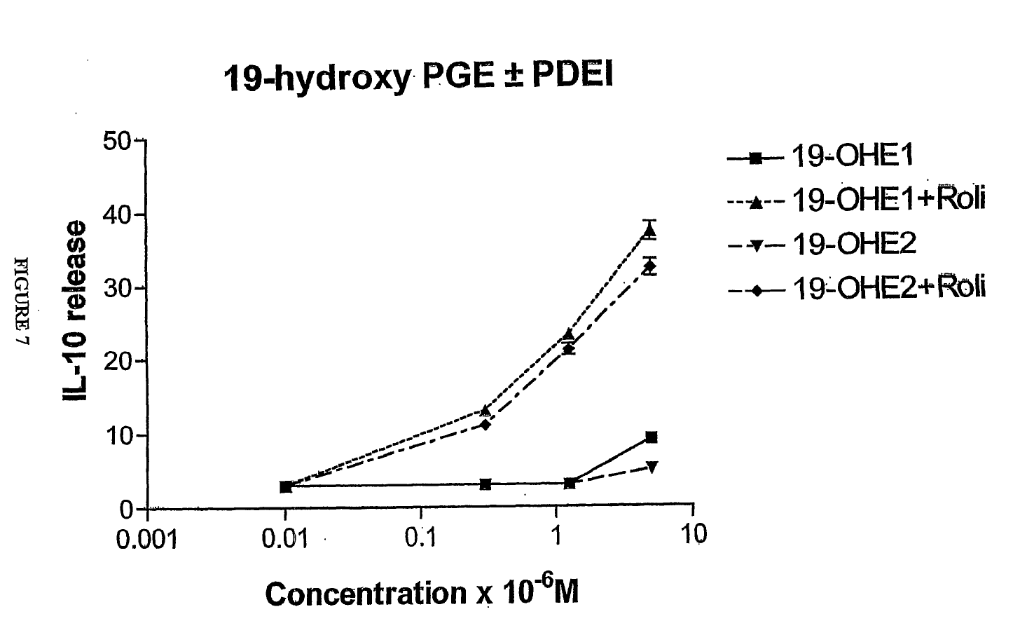

FIG. 8

A graph showing the effect of PGE1 and PGE2 on the stimulation of IL-10 in the presence and absence of rolipram.

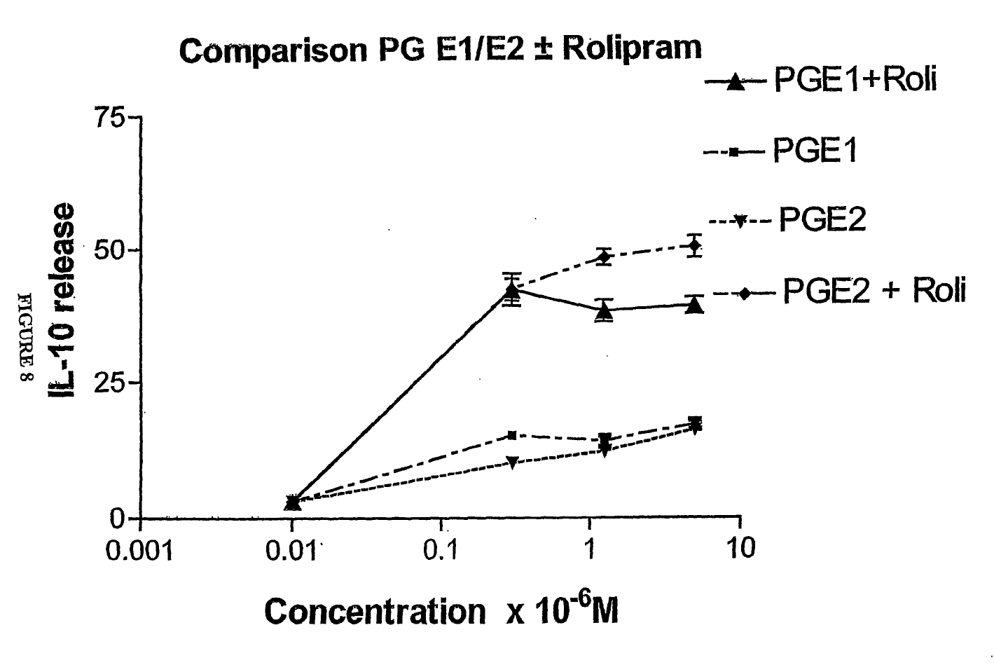

FIG. 9

A graph showing the effect of PGE and 19 hydroxy PGE on the production of phosphodiesterase IV b mRNA in the presence and absence of rolipram.

FIG. 10

Stem cell lineages for human pluripotent stem cells.

FIG. 11

FIG. 11 is a diagram showing agents which control intracellular cAMP. Open arrows are effectively lowering intracellular cAMP levels. Solid arrow is stimulation. Combinations will be synergistic.

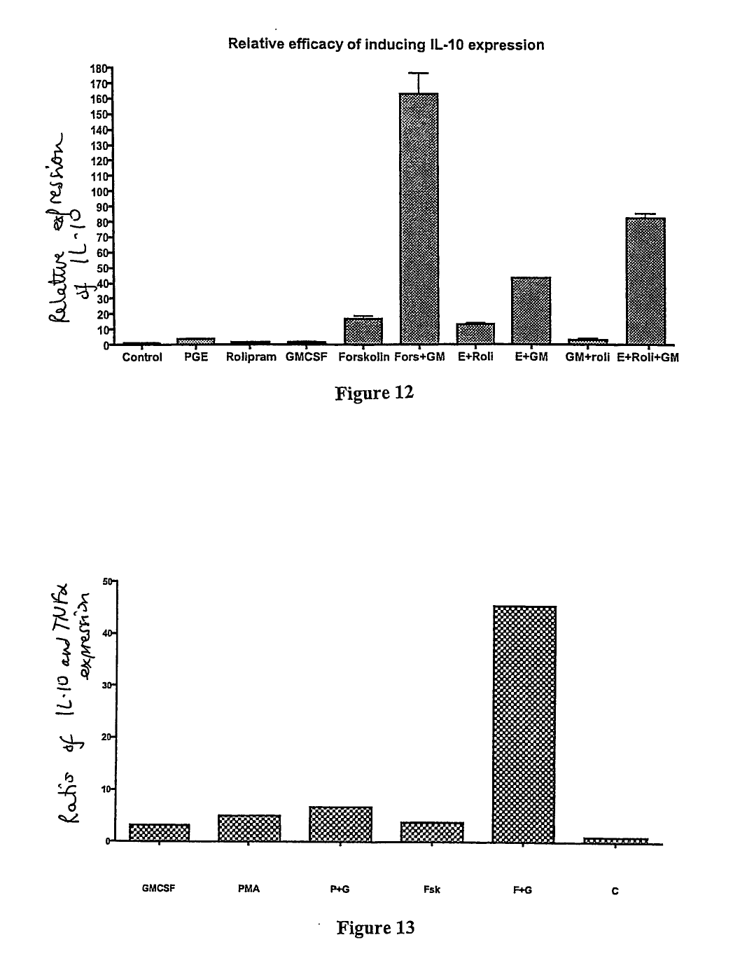

FIG. 12

FIG. 12 shows the relative efficacy of various agents in inducing IL-10 expression. See Example 4 for details.

FIG. 13

FIG. 13 shows the relative efficacy of various agents in inducing IL-10, expressed as a ratio of IL-10/TNFα mRNA expression. See Example 5 for details.

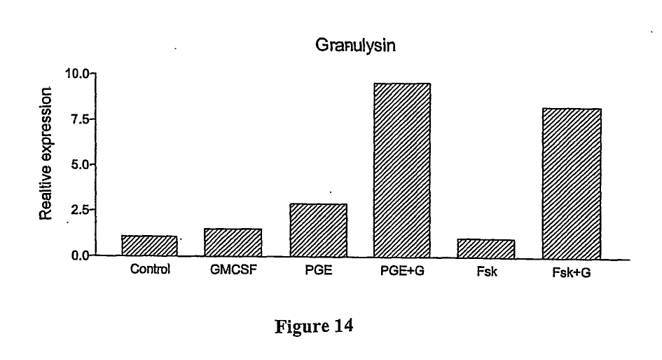

FIG. 14

FIG. 14 shows the relative efficacy-of various agents and combinations of agents in inducing granulysin mRNA expression. See Example 6 for details.

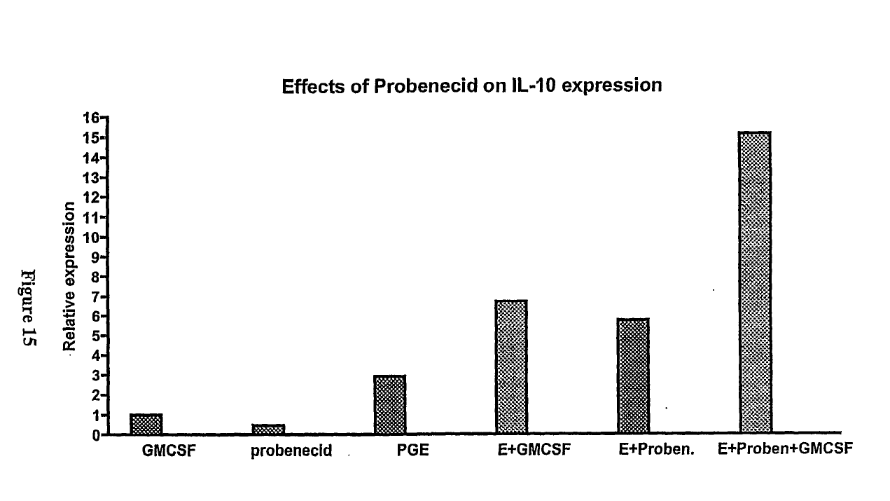

FIG. 15

FIG. 15 shows that there is a synergistic effect between a prostaglandin (PGE2) and GMCSF and probenicid on the expression of IL-10.

### EXAMPLE 1

Prostaglandin E/GMCSF Synergism for Inducing Immunological Tolerance

There is growing evidence that prostaglandins of the E series are involved in immunological tolerance. This derives from their role in oral tolerance (the ability of the immune system to distinguish pathogenic and comensal organisms), their ability to modulate cytokine ratios, and their huge concentrations in human seminal plasma where tolerance for the spermatozoon is essential.

Prostaglandins are produced at most mucosal surfaces of the body that have to accommodate beneficial or harmless bacteria and yet mount a response to pathogens. Newberry et al (1999) Nature Medicine 5, 900-906 have shown that 3A9 TCRA −/− mice expressing a T cell receptor that specifically recognises egg-white lysosyme do not mount an inflammatory response to this antigen unless prostaglandin synthesis is inhibited, in that case by inhibiting the inducible cyclooxygenase isoform COX-2. With the source of prostaglandin removed, and with exposure to the specific antigen, these mice develop a pathology resembling inflammatory bowel disease (Newberry et al (1999) supra). These experiments confirm earlier studies showing that non-steroidal anti-inflammatory drugs such as indomethacin, which have a primary effect of inhibiting prostaglandin synthesis, break tolerance (Scheuer et al (1987) Immunology 104, 409-418; Louis et al (1996) Immunology 109, 21-26).

Monocytes of the normal lamina propria have a distinct phenotype since they express CD86 but not CD80. When an inflammatory condition persists (eg inflammatory bowel disease) the monocytes express CD80 (Rugtveit et al (1997) Clin. Exp. Immunol. 104, 409-418). The resident macrophages (CD80−ve CD86+ve) are thus distinguished from the recently recruited macrophages which are CD80+ve, CD86+ve.

Monocytes are major sources of many immunological mediators, including prostaglandins and as such will alter the cytokine environment for antigen presentation. PGE has a major effect on cytokines relevant to tolerance, stimulating the tolerogenic cytokine IL-10 (Strassmann et al (1994) J. Exp. Med. 180, 2365-2370) and inhibiting IL-12 (Kraan et al (1995) J. Exp. Med 181, 775-779) which breaks tolerance. PGE will also have direct effects on the maturation of antigen-presenting dendritic cells, stimulating the production of cells that secrete increased IL-10 and diminished IL-12 (Kalinski et al (1997) Adv. Exp. Med. Biol 417, 363-367).

A further indication of the importance of prostaglandins in ensuring essential tolerance is the very high (approximately millimolar) concentrations of both PGE and 19-hydroxy PGE in human seminal plasma. Clearly, immunological tolerance for spermatozoa entering the immunogically competent, and possibly infected, female genital tract is essential for the continuation of the species and levels of prostaglandin are such that many sub-epithelial, and even lymph-node cells will be affected. In this way, evolution has ensured immunological protection for the spermatozoa.

Previous experiments (Strassmann et al (1994) supra; Kraan et al (1995) supra) have required lipopolysaccharide (LPS) to be present for PGE to stimulate IL-10 production and in addition, the message for IL-10 was delayed by approximately 12 hours, both of these factors has been puzzling. The observations of the present invention suggest that LPS may have been stimulating the expression of GMCSF, which may account for both the delay and the subsequent IL-10-expression

We now show that the major prostaglandin effects on tolerance inducing monocytes may be mediated by a synergism between a prostaglandin and GMCSF. The result of short term exposure to this combination results in a phenotype expressing greatly increased IL-10 but reduced levels of participants in antigen presentation such as CIITA and MHCII. Moreover, this change in phenotype is accompanied by enhanced expression of granulysin. This molecule has anti-microbial properties (Krensky (2000) Biochem. Pharmacol. 59, 317-320) and is normally thought of as a product of activated T cells—mediating antiviral activity that lyses infected cells (Hata et al (2001) Viral Inmunol. 14; 125-133; Ochoa et al (2001) Nature Medicine 7, 174-179; Smyth et al (2001) J. Leukos. Biol. 70, 18-29. Such an increase in innate defence molecules may compensate for the compromise of the adaptive immune system that necessarily accompanies tolerance induction. The phenotype is further characterised by a neutral effect on CD80 but a stimulation of CD86.

Experimental Details

U937 (human monocyte cell line) cells were grown in RPMI (PAA Laboratories) medium with 10% fetal calf serum added (PAA Laboratories). Cells were treated with prostaglandin E2 at 10⁻⁶ Molar with or without GMCSF with at 5 ng/ml for 4 hours. The treatment was removed and cells were cultured for a further 20 hours. Cells were pelleted and the MRNA was extracted with Tri reagent (Sigma, Poole, UK). Total RNA was obtained by addition of chloroform and subsequent isopropanol precipitation. RNA was reverse transcribed with reverse transcriptase (Applied Biosystems) and random hexamers (Applied Biosystems). Probes and primers for amplification and detection of IL-10 and a number of other molecules were designed using Primer Express (Applied Biosystems) and are as follows:

IL-10 primers
CTACGGCGCTGTCATCGAT
TGGAGCTTATTAAAGGCATTCTTCA
IL-10 probe
CTTCCCTGTGAAAACAAGAGCAAGGCC
BAX primers
CATGGAGCTGCAGAGGATGA
CTGCCACTCGGAAAAAGACCT
Bax Probe
TGCCGCCGTGGACACAGACTT
BCL2 primers
CCGGGAGGCGACCGTAGT
GGGCTGCGCACCCTTTC
BCL2 probe
CGCCGCGCAGGACCAGGA
CD80 primers
TCCACGTGACCAAGGAAGTG
CCAGCTCTTCAACAGAAACATTGT
CD80 Probe
AAGAAGTGGCAACGCTGTCCTGTGG
CD86 primers
CAGACCTGCCATGCCAATT
TTCCTGGTCCTGCCAAAATACTA
CD86 Probe
CAAACTCTCAAAACCAAAGCCTGAGTGAGC
COX-1 primers
TGGTTCGGTGTCCAGTTCCAATA
ACCTTGAAGGAGTCAGGCATGAG
COX-1 Probe
CGCAACCGCATTGCCATGGAGT
COX-2 primers
GTGTTGACATCCAGATCACATTTGA
GAGAAGGCTTCCCAGCTTTTGTA
COX-2 Probe
TGACAGTCCACCAACTTACAATGCTGACTATGG
EP2 primers
GAC CGC TTA CCT GCA GCT GTA C
TGA AGT TGC AGG CGA GCA
EP2 Probe
CCA CCC TGC TGC TGC TTC TCA TTG TCT
EP4 primers
ACGCCGCCTACTCCTACATG
AGAGGACGGTGGCGAGAAT
EP4 Probe
ACG CGG GCT TCA GCT CCT TCC T
PDE4b primers
CCTTCAGTAGCACCGGAATCA
CAAACAAACACACAGGCATGTAGTT
PDE4b Probe
AGCCTGCAGCCGCTCCAGCC
Granulysin primers
CAGGGTGTGAAAGGCATCTCA
GGAGCATGGCTGCAAGGA
Granulysin Probe
CGGCTGCCCCACCATGGC
CD14 primers
GCGCTCCGAGATGCATGT
AGCCCAGCGAACGACAGA
CD14 Probe
TCCAGCGCCCTGAACTCCCTCA
E synthase primers
CGGAGGCCCCCAGTATTG
GGGTAGATGGTCTCCATGTCGTT
E synthase Probe
CGACCCCGACGTGGAACGCT
IRAKM primers
CCT GCC TCG GAA TTT CTC T
CTT TGC CCG CGT TGC A
IRAKM probe
CAC ACC GGC CTG CCA AAC AGA A
CIITA primers
GCTGTTGTGTGACATGGAAGGT
RTGGGAGTCCTGGAAGACATACTG
CIITA Probe
CCGCGATATTGGCATAAGCCTCCCT
Class II primers
AGCCCAACGTCTCATCTGT
TCGAAGCCACGTGACATTGA
ClassII Probe
TCATCGACAAGTTCACCCCACCAGTG

Template was amplified in a Taqman 7700 machine for 40 cycles using FAM/TAMRA dyes on the probe. The Applied Biosystems Kit was used to amplify and detect ribosomal (18S) RNA as a control. After 40 cycles the Ct (related to cycle number at which signal appears) for the FAM and the 18S (VIC) were recorded and absolute relative quantitation was achieved using the formula 2^{-ΔΔCt}.

The results of this experiment are shown in FIG. 2 and show that there is a synergistic between a prostaglandin (PGE2) and GMCSF on the release of IL-10, CD-14, CD86, COX-2, and granulysin from cells of the immune system.

### EXAMPLE 2

Prostaglandin E/GMCSF Synergism for Inducing IL-10

Cells were cultured as described in Example 1 but after 4 hours medium was removed, cells were washed and the cells were cultured in medium alone for a further 48 hours. RNA was extracted from the cells as described in Example 1.

The results of this experiment are shown in FIG. 3 and show that there is a synergistic effect between a prostaglandin (PGE2) and GMCSF on the expression of IL-10, and that this phenotype is maintained 48 hours after removal of the treatment.

### EXAMPLE 3

Release of IL-10 from Monocytes in Response to PGE and GMCSF

U937 cells were grown in RPMI (PAA Laboratories) medium with 10% foetal calf serum (PAA Laboratories) added. (tells were treated with prostaglandin E2 at 10⁻⁶ Molar both with and without GMCSF at 5 ng/ml for 4 hours. The treatment was removed and cells were cultured for a further 20 hours. Medium was removed and assayed for IL-10 using a matched monoclonal antibody pair (Pharmingen) or a commercial ELISA (R&D Systems, catalogue number D1000, Abingdon, Oxford). FIG. 4 shows the release of IL-10 from monocytes in response to PGE and GMCSF.

To assay for cyclic AMP levels, wells in which cells are growing are treated with 0.01N hydrochloric acid to extract intracellular cAMP. This extract is neutralised to pH 6 and assayed for cyclic AMP in a competitive enzyme immunoassay (R&D Systems, catalogue number DE0450, Abingdon, Oxford).

### EXAMPLE 4

Effect of the Combination of PGE and Rolipram on IL-10 and IL-12 Production by U-937 (Promonocyte) Cells

U 937 (human monocyte cell line) cells were grown in RPMI (PAA Laboratories) medium with 10% fetal calf serum added (PAA Laboratories). Cells were treated with prostaglandin E 2 at 10⁻⁶ Molar or with Interferon-γ at 10 ng/ml for 24 hours.

Rolipram at 1 μg/ml and indomethacin at 10 μM was present in all wells. Cells were pelleted and the mRNA was extracted with Tri reagent (Sigma, Poole, UK). Total RNA was obtained by addition of chloroform and subsequent isopropanol precipitation. RNA was reverse transcribed with reverse transcriptase (Applied Biosystems) and random hexamers (Applied Biosystems). Probes and primers for IL-10 and IL-12 (p35) were designed using Primer Express (Applied Biosystems) and were as follows:

IL-12 p35 primers
CCACTCCAGACCCAGGAATG
TGTCTGGCCTTCTGGAGCAT
IL-12 probe
TCCCATGCCTTCACCACTCCCAA
IL-10 primers
CTACGGCGCTGTCATCGAT
TGGAGCTTATTAAAGGCATTCTTCA
IL-10 probe
CTTCCCTGTGAAAACAAGAGCAAGGCC

Template was amplified in a Taqman 7700 machine for 40 cycles using FAM/TAMRA dyes on the probe. The Applied Biosystems Kit was used to amplify and detect ribosomal (18S) RNA as a control. After 40 cycles the Ct (related to cycle number at which signal appears) for the FAM and the 18S (VIC) were recorded and absolute relative quantitation was achieved using the formula 2^{-ΔΔCt}.

The results of this experiment are described in the legend to FIG. 5. They show that there is a synergistic between a prostaglandin (PGE2) and a PDE inhibitor (rolipram) on the release of IL-10 from cells of the immune system and that there is a marked stimulation of IL-10 and inhibition of IL-12 in cells of the immune system when a prostaglandin (PGE2) and a PDE inhibitor (rolipram) are used in combination.

### EXAMPLE 5

Stimulation of IL-10 Production is Achieved with or without LPS

U 937 cells were grown in RPP (PAA Laboratories) medium with 10% fetal calf serum added (PAA Laboratories). 2×10⁶ cells per flask were treated with prostaglandin E₂ at 10⁻⁶ Molar or with Rolipram (4×10⁻⁶) for 24 hours. Medium was removed at 20 hours and analysed by ELISA. A capture antibody (Pharmingen) was coated onto 96 well plates and culture medium was added each well. A standard curve was created with recombinant IL-10 protein. After incubation and washing, a biotin labelled monoclonal antibody (Pharmingen) was added and following incubation and washing, peroxidase labelled streptavidin was added. After washing a tetramethyl benzidine substrate was added and colour developed in proportion to IL-10 in the original sample/standard. Colour was read using a plate photometer (Labsystems, Multiskan). Mean concentrations (N=3) in controls with no lipopolysaccharide (LPS) were 38.2 pg/ml and in the presence of LPS (100 nM) they were 43.9 prostaglandin/ml.

After the incubation (20 hours), cells were pelleted and the mRNA was extracted with Tri-reagent (Sigma, Poole, UK). Total RNA was obtained by addition of chloroform and subsequent isopropanol precipitation. RNA was reverse transcribed with reverse transcriptase (Applied Biosystems) and random hexamers (Applied Biosystems). Probes and primers for IL-10 and IL-12 (p35) were designed using Primer Express (Applied Biosystems) and were as follows:

IL-12 p35 primers
CCACTCCAGACCCAGGAATG
TGTCTGGCCTTCTGGAGCAT
IL-12 probe
TCCCATGCCTTCACCACTCCCAA
IL-10 primers
CTACGGCGCTGTCATCGAT
TGGAGCTTATTAAAGGCATTCTTCA
IL-10 probe
CTTCCCTGTGAAAACAAGAGCAAGGCC

Template was amplified in a Taqman 7700 machine for 40 cycles using FAM/TAMRA dyes on the probe. The Applied Biosystems kit was used to amplify and detect ribosomal (18S) RNA (using VIC/TAMRA dyes) as an internal control in the same reaction tube. After 40 cycles the Ct (related to cycle number at which signal appears) for the FAM and the 18S (VIC) were recorded and absolute relative quantitation was achieved using the formula 2^{-ΔΔCt} where Δ refers to the difference between the FAM and VIC signal related to an standard comparator included in each run.

### EXAMPLE 6

The effect of PGE1, PGE2, 19 hydroxy PGE1 and 19 hydroxy PGE2 on the stimulation of IL-10 in the presence and absence of rolipram was investigated as described above in Example 5. IL-10 levels were measured using an ELISA assay (R&D Ltd, Oxford). Measurement was performed according to the manufacturer's instructions. Results are shown in FIGS. 7 and 8.

### EXAMPLE 7

The mRNA for phosphodiesterase IV-b was measured as described in Example 5 above. mRNA was extracted after four hours of incubation. The concentration of the PGE was 1×10⁻⁶ and that of the 19-hydroxy PGE₂ was 5×10⁻⁶. The following primers and Taqman probe were used for quantitation of PDE IV b mRNA.

Forward
CCTTCAGTAGCACCGGAATCA
Reverse
CAAACAAACACACAGGCATGTAGTT
Probe
AGCCTGCAGCCGCTCCAGCC

Results are shown in FIG. 9. An increase in PDE activity follows both PGE and 19-hydroxy PGE application, which appears to be a direct negative feedback to reduce the effect of the stimulus. Use of a PGE and a type IV selective PDE inhibitor increases PDE message levels even further, but then the synthesised phosphodiesterase is nullified by the presence of the inhibitor.

### EXAMPLE 8

Relative Efficacy of Various Agents which Raise cAMP Levels in Monocyte Cells in Inducing IL-10

Experimental Details

U937 (human monocyte cell line) cells were grown in RPMI (PAA Laboratories) medium with 10% fetal calf serum added (PAA Laboratories). Cells were treated with prostaglandin E2 at 10⁻⁶ Molar, Rolipram 10⁻⁶ Molar, Forskolin 50×10⁻⁶ Molar with or without GMCSF at 5 ng/ml for 48 hours. Cells were pelleted and the mRNA was extracted with Tri reagent (Sigma, Poole, UK). Total RNA was obtained by addition of chloroform and subsequent isopropanol precipitation. RNA was reverse transcribed with reverse transcriptase (Applied Biosystems) and random hexamers (Applied Biosystems). Probes and primers for amplification and detection of IL-10 were designed using Primer Express (Applied Biosystems) and are as follows:

IL-10 primers
CTACGGCGCTGTCATCGAT
TGGAGCTTATTAAAGGCATTCTTCA
IL-10 probe
CTTCCCTGTGAAAACAAGAGCAAGGCC

See FIG. 12.

### EXAMPLE 9

Relative Efficacy of Various Agents which Raise cAMP Levels in Monocyte Cells in Inducing IL-10 Compared to TNFα

As for Example 4 but MRNA for TNFα is also included.

PMA (2×10⁻⁷ M) was used as an alternative differentiating agent and although IL-10 was increased by PMA differentiation, TNFα (a pro-inflammatory and antitolerogenic agent) was also increased. Differentiation with Forskolin and GMCSF did not appreciably raise TNFα. Data is shown as the ratio of IL-10 mRNA/TNFα mRNA. P=PMA=Phorbol myristoyl acetate; F=Fsk=Forskolin g=GMCSF, C=vehicle control.

TNFα Primers
GGAGAAGGGTGACCGACTCA
TGCCCAGACTCGGCAAAG
TNFα probe
CGCTGAGATCAATCGGCCCGACTA

See FIG. 13.

### EXAMPLE 10

Relative Efficacy of Various Agents which Raise cAMP Levels in Monocyte Cells in Inducing Granulysin

As for Example 4 but mRNA for granulysin was measured using the primers listed in Example 1 (see FIG. 14).

G=GMCSF; FSK=Forskolin

### EXAMPLE 11

Prostaglandin E/GMCSF/Probenecid Synergism for Inducing IL-10

Cells were cultured as described in Example 1 but after 20 hours medium was removed, cells were washed and RNA was extracted from the cells as described in Example 1.

The results of this experiment are shown in FIG. 15 and show that there is a synergistic effect between a prostaglandin (PGE2) and GMCSF and probenecid on the expression of IL-10.

E=PGE2

### EXAMPLE 12

Pre-Tolerisation with Cytokines and Cross-Transplantation Studies

Experimental Design

General Description

Tissue (skin) from one inbred strain of mice (C57BL/6) was transplanted to another inbred strain which is known to be genetically/immunologically distinct (BALB/c). The pre-tolerisation regime requires the isolation of leukocytes from the donor mice. These leukocytes are then mixed with a PGE anulogue (IL-6,16-dimethyl PGE2) and murine GM-CSF. This mixture is then injected into the peritoneal cavity of the recipient mice, 48 hours prior to skin-grafting and 24 hours prior for a total of two injections. The hypothesis is that this regime will pre-tolerise the recipient mice so that donated skin-grafts will “sake” i.e. not be rejected. Mice will be observed and transplanted areas will be inspected daily for signs of rejection.

Upon rejection animals were sacrificed and transplanted areas biopsied, and if rejection had not occurred within 20 days animals were sacrificed and transplanted areas biopsied. Histology is performed on biopsied areas and stained with H&E.

Animal Criteria

Donors:
   Species/strain: mice: C57BL/6
   Number and sex: 18 Females+2 spares
   Age: 6-8 weeks old
   Weight: Commensurate with age
   Vendor: Simonsen Laboratories or Charles River
   Acclimation: 3 days
Recipients:
   Species/strain: mice: BALB/c
   Number and sex: 9 Females+2 spares
   Age: 6-8 weeks
   Weight: Commensurate with age
   Vendor: Simonsen laboratories, or Charles River

Acclimation: 3 days

Group Assignments and Dose Levels

| Group | | | Graft | Cocktail | Leukocyte |
|---|---|---|---|---|---|
| No. | No | Procedure | type | treatment | treatment |
| 1 | 6 | Mice cross transplanted | C57 to | Yes | Yes |
| | | and treated | BALB/c | | |
| 2 | 6 | Mice cross transplanted | C57 to | No | NO |
| | | and not treated | BALB/c | | |
| 3 | 2 | Mice holograpically | BALB/c | Yes | Yes |
| | | transplanted treated | to | | |
| | | controls | BALB/c | | |
| 4 | 2 | Mice holograpically | BALB/c | No | No |
| | | non treated controls | to | | |
| | | | BALB/c | | |
| 5 | 2 | Cocktail alone to check | C57 to | Yes | No |
| | | tolerance (no leukocytes) | BALB/c | | |

| | Dose Level |
|---|---|
| Agent | μg/kg/dose |
| 16,16-dimethyl PGE2 | 400 |
| GM-CSF | 5.0 |

Routes and Schedules

A cocktail of GM-CSF+Prostaglandin+Leukocytes was injected intraperitoneally 48 hours and 24 hours prior to skin grafting.

Dosing Procedure/s

Doses were administered via intraperitoneal injection in a volume of approximately 150 μL. The animals were temporarily restrained by scruffing for dosing, but were not sedated. Disposable sterile syringes were used for each animal/dose.

Leukocyte+Cytokine Cocktail

Leukocyte Preparation

For preparation of leukocytes from mouse blood an alternative to lymphoprep or Ficoll sedimentation. Centrifuge 0.2 ml whole blood from donor mouse (the one from which the transplant is taken) to pellet all cells. Remove supernatant carefully and resuspend cells in 0.2 ml of red blood cell lysis buffer (Sigma cat no R7757 p1178 of the 2004 catalogue). After 1 minute add 3 ml of buffered physiological saline (PBS). Centrifuge 500 g for 7 minutes and thoroughly resuspend the pellet in 100 to 200 μl PBS. This lyses most of the red blood cells but many remain and the solution is quite red. This does not matter since the idea is just to get rid of the majority of the red blood cells.

Cocktail Preparation for Injection

Cocktail of GMCSF and dimethyl PGE2 was mixed immediately before injection IP in minimum of solution—say 100 μl.

The GM-CSF and prostaglandin cocktail in 100 μl was mixed with 50 μl of the cell preparation and injected intraperitoneally on two successive days.

Transplant Procedure

Mice were anaesthetized. Two 1.0 in² pieces of full-thickness trunk skin were harvested from 6- to 8-week-old donor mice from each of their flanks. The recipient graft area and donor skin were prepared by cleaning with Betadine and 70% ethanol. One graft per recipient animal was sutured without undue stress on the left thorax of 6- to 8-week-old recipients. Allografts were impregnated with antibiotic ointment. Rejection is defined as graft necrosis greater than 90% of graft area. After surgery, mice will be kept in individual cages.

In-Life Observations and Measurements

Health Observations

Animals appearing ill were brought to the attention of the study director and any animals that show pronounced effects were removed from the study.

Animals were observed within their cages at least once daily throughout the study. Each animal were observed for changes in general appearance and behavior. Any abnormal observation were reported to the study director.

Graft Observations

The skin graft for each animal was observed for necrosis, coloration, hydration, capillary refill time, and skin tension.

Body Weights

Body weights were measured prior to the first dose and weekly thereafter.

Materials and Methods

Test/Control Article Information

Route

The intraperitoneal route was chosen because this route has proven effective for similar studies based on literature searches.

Identification

Prostaglandin

16,16-dimethyl PGE2 used at 400 μg/kg
5 mg pack (in triacetin) approx
Catalog number 14750.1
Use at 400 μg (microgrammes) per kilogram body weight
Source: Cayman Chemicals
1180E Ellsworth Road
Ann Arbor
Michigan 48108
www.caymanchem.com
GM-CSF:
Murine Granulocyte macrophage colony stimulating factor
From Peprotech
www.Peprotech.com
Catalog number 315-03
Freely soluble in aqueous solution use at 5 μg (microgram) per kilogram body weight
   Results and Conclusions

The following observations were made at 15 days (for Group 1), 14 days (for Group 2) and 13 days (for Groups 3, 4 and 5). Thus, the treatment group (Group 1) is one day further advanced than the equivalent group with no treatment (Group 2).

Group 1:

#5518—normal in center, edges appear necrotic
#5522—normal in center, edges appear necrotic
#5528—lost skin graft, remaining site is scabbed
#5530—10-20% necrotic, moisturized, mild tension
#5531—2040% necrotic, dehydrated, puffy
#5535—2040% necrotic, moisturized, moderate tension
   Gp 2:
#5513—40-60% necrotic, dehydrated, mild tension
#5520—10-20% necrotic, moisturized, moderate tension
#5523—10-20% necrotic, moisturized, moderate tension
#5527—10-20% necrotic, moisturized, moderate tension
#5532—10-20% necrotic, moisturized, moderate tension
#5536—10-20% necrotic, moisturized, moderate tension
   Gp3:
#5519—normal skin
#5521—normal skin
   Gp 4:
#5515—normal skin
#5517—normal skin
   Gp 5:
#5531—20-40% necrotic, moisturized, mild tension
#5534—10-20% necrotic, moisturized, moderate tension

The results provide evidence that rejection has been delayed in the treatment group (Group1) compared to the non-treatment group (Group 2).

### EXAMPLE 13

Pre-Tolerisation of Patient Undergoing Stem Cell Treatment of Diabetes Mellitus

Nestin-positive islet-derived precursor cells are isolated as described in Lechner et al (2002) Biochem. Biophys. Res. Comm. 293, 670-674. They are combined in a pessary with PGE2 and rolipram and/or GMCSF and the pessary inserted into the vagina of the female patient in order to tolerise the patient to the cells. Ten days later, 10⁶-10⁷ of the nestin-positive islet-derived precursor cells are administered to the patient using the Edmonton protocol protocol. In brief, the cells are injected into the hepatic portal vein of the patient from which they are taken to the liver where they form an insulin-producing islet.

### EXAMPLE 14

Pre-Tolerisation of Patient Undergoing Stem Cell Treatment of Parkinson's Disease

Human embryonic stem cells from an ethically approved donor source are differentiated into dopamine-producing neural cells by co-culture with PA6 cells, a stromal cell line derived from skull bone marrow. These are combined with PGE2 and rolipram and/or GMCSF and formulated into a pessary. The patient is administered the pessary.

Once tolerance to the stem cells is achieved, the neural cells are introduced into one of the accessible ventricles or portal veins from where they migrate to the correct site in the substantia nigra of the patient and integrate.

1. A method of inducing tolerance to a therapeutic cell in a patient who is to be administered subsequently a therapeutic amount of the said therapeutic cell or a precursor thereof, the method comprising administering to the patient (a) a tolerising cell sharing the same antigenic characteristics as the therapeutic cell, or an antigen found thereon or a derivative of said antigen, and (b) an agent which raises the effective cAMP concentration in a monocyte cell.

2. A method of reducing the risk of rejection of a transplant in a patient in need of transplantation of a therapeutic cell for cell or tissue regeneration, the method comprising administering to the patient prior to the transplant (a) a tolerising cell sharing the same antigenic characteristics as the therapeutic cell which therapeutic cell is, or is able to differentiate into, the cell or tissue to be regenerated, or an antigen found thereon or a derivative of said antigen, and (b) an agent which raises the effective cAMP concentration in a monocyte cell.

3. A method of treating a patient in need of cell or tissue regeneration, the method comprising administering to the patient (a) a tolerising cell sharing the same antigenic characteristics as a therapeutic cell to be administered subsequently which therapeutic cell is, or is able to differentiate into, the cell or tissue to be regenerated, or an antigen found thereon or a derivative of said antigen, (b) an agent which raises the effective cAMP concentration in a monocyte cell in an amount to induce tolerance to the said therapeutic cell, and subsequently administering to the patient (c) a therapeutic amount of the said therapeutic cell.

4. A method according to claim 3 wherein in step (a) a cell is administered to the patient.

5. A method according to claim 4 wherein the tolerising cell in step (a) and the therapeutic cell in step (c) are derived from the same parent embryonic stem cell.

6. A method according to claim 1 wherein the patient is additionally administered granulocyte-macrophage colony stimulating factor (GMCSF) or a derivative thereof.

7. A method according to claim 3 wherein the patient is suffering from a degenerative disease or disorder.

8. A method according to claim 7 wherein the degenerative disease or disorder is selected from the group consisting of diabetes, stroke, Parkinson's disease, ALS (Lou Gehrig's disease), spinal cord injury, heart attack, cardiac ischaemia, congestive heart failure, hepatitis, cirrhosis, cancer, immunodeficiency, osteoporosis, osteoarthritis, macular degeneration, bum, wounds, muscular dystrophy and multiple sclerosis.

9. A method according to claim 1 wherein (a) the tolerising cell or an antigen found thereon or a derivative of said antigen, and (b) the agent which raises the effective cAMP concentration in a monocyte cell are administered together.

10. A method according to claim 9 wherein GMCSF is administered at the same time as (a) the tolerising cell, or an antigen found thereon or a derivative of said antigen, and (b) the agent which raises the effective cAMP concentration in a monocyte cell.

11. A method according to claim 1 wherein (a) the tolerising cell or an antigen found thereon or a derivative of said antigen is administered after administration of (b) the agent which raises the effective cAMP concentration in a monocyte cell and, if used, GMCSF or a derivative thereof.

12-24. (canceled)

25. A composition for inducing tolerance to a therapeutic cell in a patient who is to be administered subsequently a therapeutic amount of the said therapeutic cell or a precursor thereof, the composition comprising (a) a tolerising cell sharing the same antigenic characteristics as the therapeutic cell, or an antigen found thereon or a derivative of said antigen, (b) an agent which raises the effective cAMP concentration in a monocyte cell, and optionally, (c) granulocyte-macrophage colony stimulating factor (GMCSF) or a derivative thereof.

26. A therapeutic system for inducing tolerance to a therapeutic cell in a patient who is to be administered subsequently a therapeutic amount of the said therapeutic cell or a precursor thereof, the therapeutic system comprising (a) a tolerising cell sharing the same antigenic characteristics as the therapeutic cell, or an antigen found thereon or a derivative of said antigen, (b) an agent which raises the effective cAMP concentration in a monocyte cell, and optionally, (c) granulocyte-macrophage colony stimulating factor (GMCSF) or a derivative thereof.

27. A kit of parts for inducing tolerance to a therapeutic cell in a patient who is to be administered subsequently a therapeutic amount of the said therapeutic cell or a precursor thereof, the kit comprising (a) a tolerising cell sharing the same antigenic characteristics as the therapeutic cell, or an antigen found thereon or a derivative of said antigen, (b) an agent which raises the effective cAMP concentration in a monocyte cell, and optionally, (c) granulocyte-macrophage colony stimulating factor (GMCSF) or a derivative thereof.

28. A method according to claim 1 wherein the agent which raises the effective cAMP concentration in a monocyte cell is any one or more of a prostaglandin or agonist thereof, a β-adrenergic agent, a blocker of cAMP export from the cell, forskolin or a derivative thereof, a cAMP phosphodiesterase inhibitor, a cAMP analogue, or cholera toxin or a derivative or fragment thereof.

29. A method according to claim 28 wherein the blocker of cAMP export from the cell is probenicid or progesterone.

30. A method according to claim 28 wherein the cAMP analogue is Sp-adenosine cyclic 3′, 5′-cyclic monophosphorothioate or 8-bromoadenosine 3′, 5′ monophosphate or dibutyryl cAMP.

31. A method according to claim 28 wherein the prostaglandin or agonist thereof stimulates cAMP production in a monocyte.

32. A method according to according to claim 28 wherein the prostaglandin or agonist thereof is any one of a prostaglandin E, dinoprostone, gemeprost, misoprostol, alprostadil, limaprost, butaprost, 11-deoxy PGE1, AH23848, AH13205, or a 19-hydroxy PGE.

33. A method according to claim 6 wherein the GMCSF is human GMCSF having the amino acid sequence as defined in FIG. 1, or naturally occurring variants thereof.

34. A method according to claim 33 wherein the GMCSF is sargramostim.

35. A method according to claim 1 comprising administering a monocyte chemotactic agent to the patient.

36. A method according to claim 35 wherein the monocyte chemotactic agent is MCP-1 or MIP-1α.

37. A method according to claim 1 further comprising administering a PDE inhibitor to the patient.

38. A method according to claim 28 wherein the PDE inhibitor is any one of 3-isobutyl-1-methylxanthine (IBMX), pentoxifylline (3,7-dihydro-3,7-dimethyl-1-(5-oxohexyl)-1H-purine-2,6-dione), rolipram (4-[3-cyclopentyloxy-4-methoxyphenyl]-2-pyrrolidinone), CP80 633, CP102 995, CP76 593, Ro-20-1724 (4-[3-butoxy-4-methoxybenzyl]-2-imidazolidinone), theophylline, or denbufylline (1,3-di-n-butyl-7-(2-oxopropyl)-xanthine).

39. A method according to claim 38 wherein the PDE inhibitor is selective for type IV PDE.

40. A method according to claim 39 wherein the PDE inhibitor selective for type IV PDE is any one of rolipram (4-[3-cyclopentyloxy-4-methoxyphenyl]-2-pyrrolidinone), CP80 633, CP102 995, CP76 593, Ro-20-1724 (4-[3-butoxy-4-methoxybenzyl]-2-imidazolidinone), denbufylline (1,3-di-n-butyl-7-(2-oxopropyl)-xanthine, or CDP840, RP73401 or RS33793.

41. A pharmaceutical composition comprising the composition according to claim 25 and a pharmaceutically acceptable carrier, diluent or excipient.

42. (canceled)

43. A therapeutic system according to claim 26 further comprising a therapeutic cell which is, or is able to differentiate into, a cell or tissue to be regenerated.

44-45. (canceled)

46. A composition according to claim 25 wherein the agent which raises the effective cAMP concentration in a monocyte cell is any one or more of a prostaglandin or agonist thereof, a β-adrenergic agent, a blocker of cAMP export from the cell, forskolin or a derivative thereof, a cAMP phosphodiesterase inhibitor, a cAMP analogue, or cholera toxin or a derivative or fragment thereof.

47. A therapeutic system according to claim 26 wherein the agent which raises the effective cAMP concentration in a monocyte cell is any one or more of a prostaglandin or agonist thereof, a β-adrenergic agent, a blocker of cAMP export from the cell, forskolin or a derivative thereof, a cAMP phosphodiesterase inhibitor, a cAMP analogue, or cholera toxin or a derivative or fragment thereof.

48. A kit of parts according to claim 27 wherein the agent which raises the effective cAMP concentration in a monocyte cell is any one or more of a prostaglandin or agonist thereof, a β-adrenergic agent, a blocker of cAMP export from the cell, forskolin or a derivative thereof, a cAMP phosphodiesterase inhibitor, a cAMP analogue, or cholera toxin or a derivative or fragment thereof.

49. A kit of parts according to claim 27 further comprising a therapeutic cell which is, or is able to differentiate into, a cell or tissue to be regenerated.

50. A composition according to claim 25 wherein the GMCSF is present and is a human GMCSF having the amino acid sequence as defined in FIG. 1, or naturally occurring variants thereof.

51. A therapeutic system according to claim 26 wherein the GMCSF is present and is a human GMCSF having the amino acid sequence as defined in FIG. 1, or naturally occurring variants thereof.

52. A kit of parts according to claim 27 wherein the GMCSF is present and is a human GMCSF having the amino acid sequence as defined in FIG. 1, or naturally occurring variants thereof.
